(19) United States
(12) Patent Application Publication
(10) Pub. No.: US 2004/0265799 A1
(43) Pub. Date: Dec. 30, 2004

(54) Human-virus homologous sequences and uses thereof

(75) Inventors: Amit Novik, Tel Mond (IL); Eddo Kim, Raanana (IL); Erez Levanon, Petach Tikva (IL); Yossi Kliger, Rishon LeZion (IL)

Correspondence Address: Martin Moynihan, c/o ANTHONY CASTORINA, SUITE 207, 2001 JEFFERSON DAVIS HIGHWAY, ARLINTON, VA, 22202, US

(73) Assignee: Compugen Ltd.

(21) Appl. No.: 10/873,314

(22) Filed: Jun. 23, 2004

Related U.S. Application Data
(60) Provisional application No. 60/539,125, filed on Jan. 27, 2004.
(60) Provisional application No. 60/480,752, filed on Jun. 24, 2003.

(51) Int. Cl. C12Q 1/70; C07H 21/02

(52) U.S. Cl. 435/5; 536/23.1

(57) ABSTRACT

Human-viral homologs having defined properties and/or functions, for particular therapeutic and/or diagnostic applications or uses. The homologs according to the present invention also encompass many novel sequences, as well as previously known sequences for which no diagnostic and/or therapeutic application, and/or a different diagnostic and/or therapeutic application, has been taught or suggested in the background art.

### RELATED APPLICATIONS

This application claims priority from U.S. Provisional Patent Application No. 60/539,125 filed on Jan. 27, 2004, and U.S. Provisional Patent Application No. 60/480,752 filed on Jun. 24, 2003, which are hereby incorporated by reference as if fully set forth herein.

### FIELD OF THE INVENTION

The present invention relates to nucleotide and amino acid sequences, and in particular to such sequences which are homologous between humans and viruses, as well as to uses thereof.

### BACKGROUND OF THE INVENTION

Viruses are obligate intracellular parasites and, as such, use many normal cellular pathways and components during their replication cycle. For example, viruses adopt host genes in order to increase their survival chances within their hosts. Viruses use different strategies to fight the host defense system, and study of these strategies already facilitates a deeper understanding of tumorigenesis and immune defense mechanism. Large DNA viruses may contain up to a few hundred open reading frames. (ORFs). Among the proteins they encode, one can distinguish between those that have essential viral functions, and those that are involved in direct interaction with the host. The publication of the draft of the human genome and conceptual translated products (Lander et al. 2001) enables scientists to conduct a comprehensive assessment of homologous proteins between a vertebrate genome and viral ORFs. Alba and colleagues (Holzerlandt R, Orengo C, Kellam P, Alba M M. Identification of New Herpesvirus Gene Homologs in the Human Genome. Genome Res 2002 12,1739-48) have identified herpes virus/human homologs. Their study was undertaken by searching the set of conceptual and known protein sequences derived from the public Human Genome Project (Lander et al. 2001) against herpesvirus protein sequences in the virus database VIDA (Alba et al. 2001b) using two different sequence-similarity search methods. The first method was based on PSSMs derived from predefined viral protein motifs in VIDA. The second used BLAST-based pairwise sequence comparisons with the collection of singleton viral proteins and a representative set of viral proteins that share <95% sequence identity. The analysis shows that 13% of the herpesvirus proteins have clear sequence similarity to products of the human genome, and that different human herpesviruses vary in their numbers of human homologs.

1.1 Viral encoded oncogenes—The first animal tumor virus was discovered in 1909 in chickens, which are subject to infections that cause connective-tissue tumors, or sarcomas. The infectious agent was characterized as a virus—the Rous's sarcoma virus (named after its discoverer Peyton Rous), which is now known to be a retrovirus. When a radioactive DNA copy of the viral src gene sequence was used as a probe to search for related sequences by DNA-DNA hybridization, it was found that the genomes of normal vertebrate cells contain a sequence that is closely similar, but not identical, to the src gene of the Rous sarcoma virus. This normal cellular counterpart of the viral src gene (v-src) is called c-src (or just src). It is the proto-oncogene corresponding to the oncogene v-src. Evidently, the gene has been picked up accidentally by the retrovirus from the genome of a previous host cell but has undergone mutation in the process. The result is a perturbed gene function that leads to cancer and so brings the gene, and the virus that carries it, to the scientist's attention. A large number of other oncogenes have been identified in other retroviruses and analyzed in similar ways. Each has led to the discovery of a corresponding proto-oncogene that is present in every normal cell (reviewed in Molecular Biology of the Cell, 3rd edn. Part IV. Cells in Their Social Context Chapter 24. Cancer).

1.2 Viral encoded proteins that block the function of Tumor Suppressor Genes—Papillomaviruses, which are dsDNA viruses, have to be able to commandeer the host cell's DNA synthesis machinery, and the viral genes that have this function can act as oncogenes. They are called the E6 and E7 genes of the papillomavirus. The mechanism of action is apparently simple: these viral proteins bind to the protein products of two key tumor suppressor genes of the host cell, putting them out of action and so permitting the cell to replicate its DNA and divide. One of these host proteins is Rb: by binding to it, the viral protein (E7) prevents it from binding to its normal associates in the cell. The other tumor suppressor gene product that the viral proteins serve to inactivate is called p53. Like Rb, it plays a part in the development of many types of cancer, not only those dependent on viruses (reviewed in Molecular Biology of the Cell, 3rd edn. Part IV. Cells in Their Social Context Chapter 24. Cancer).

1.3 Viruses modulate the immune response.—Virally encoded ‘biopharmaceuticals’—chemokines and chemokine binding proteins—demonstrate the effectiveness of blocking a carefully selected group of chemokine receptors and how the local immune response can be changed from one dominated by Th1 cells to one dominated by Th2 cells by targeting specific chemokine receptors [reviewed in Morten Lindow, Hans Rudolf Luttichau and Thue W. Schwartz. Viral leads for chemokine-modulatory drugs. (2003) TRENDS in Pharmacological Sciences 24, 126-130]. The state of the art of the field of viral encoded chemokines, cytokines and their binding proteins is described in two recent reviews (Johnston J B, McFadden G. Poxvirus immunomodulatory strategies: current perspectives. J Virol. June 2003 ;77(11):6093-100; Seet B T, Johnston J B, Brunetti C R, Barrett J W, Everett H, Cameron C, Sypula J, Nazarian S H, Lucas A, McFadden G. Poxviruses and immune evasion. Annu Rev Immunol. 2003;21:377-423.)

1.4 Viruses modulate host cell metabolism.—See: Shugar D. Viral encoded enzymes of nucleic acid metabolism and their role in the development of antiviral agents. Prog Clin Biol Res 1982;102 Pt C: 127-38

The above functions of viral homologs also indicate that there may be potential applications of such viral homologs as therapeutic or diagnostic entities. Unfortunately, the background art does not teach or suggest a systematic method for determining the potential functionality of such homologs, nor does it teach or suggest such a method for determining suitable applications of such viral homologs as therapeutic or diagnostic entities.

### SUMMARY OF THE INVENTION

The background art does not teach or suggest a large body of human-viral homologs (homologous sequences) that have defined properties and/or functions. The background art also does not teach or suggest particular therapeutic and/or diagnostic applications or uses of such human-viral homologs.

The present invention overcomes these disadvantages of the background art by providing a large group of human-viral homologs that have such defined properties and/or functions, and as such, are useful for particular therapeutic and/or diagnostic applications or uses. This group also encompasses many novel sequences, as well as previously known sequences for which no diagnostic and/or therapeutic application, and/or a different diagnostic and/or therapeutic application, has been taught or suggested in the background art.

This group of homologs (homologous nucleic acid and/or amino acid sequences) was developed according to a computational platform according to the present invention, which provides an overall mechanism for determining sequences having the desired properties and/or functions. This information may then optionally be used for determining sequences that are suitable for particular therapeutic and/or diagnostic applications or uses. Therapeutic applications or uses may also optionally include being used as a drug and/or antibody target, for example. Therefore, the present invention also encompasses antibodies capable of binding to, and optionally also being elicited by, at least one epitope on a protein or peptide human-virus homolog. Also, the homologs according to the present invention may optionally be used as an entire sequence or as fragments thereof, such as oligonucleotides and/or peptides, and/or nucleic acid fragments and/or partial proteins or protein fragments, for example.

By “local homology” it is meant the percentage of similar amino acid residue in the overlapping region.

A “similar amino acid residue” is an amino acid residue a substitution matrix (used by algorithms for detecting alignment between proteins, e.g. Blosum 62) gives a non-negative score for its replacement for the query amino acid residue.

The “overlapping region” is the region that algorithms for finding local alignment, like BLAST, align to each other.

A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

By “treating” is meant alleviating or diminishing a symptom associated with a disease or a condition. Preferably, treating cures, e.g., substantially eliminates, and/or substantially decreases, the symptoms associated with the diseases or conditions of the present invention.

The phrase “immune disorder” refers to inflammatory diseases (e.g., inflammatory diseases associated with hypersensitivity), autoimmune diseases, infectious diseases, graft rejection diseases and allergic diseases.

Examples of autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases. By “cancer” is meant diseases associated with abnormal (hyper) cell proliferation. The term encompasses primary cancers as well as metastatic cancers. Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; malignant lymphoma, such as Birkitt's Non-Hodgkin's; Lymphoctyic leukemia, such as acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

The computational platform according to the present invention is based upon a deep analysis of the transcriptome and proteome, which is based on the human genome, the mRNA, and the ESTs of genbank version 133 (example 1) and version 136 (example 2), to find more therapeutic proteins and drug target genes as described above. The computational platform was used to identify proteins that can be used as therapeutic proteins for treating immune system disorders, cancer-related diseases, and viral infections, such as described hereinabove. Furthermore, the computational platform was used to identify genes that may serve as targets for anti-viral drugs.

The various analyses performed by the computational platform are described in greater detail below, according to different functions and methods used for these analyses.

1. Therapeutic Proteins that Modulate the Immune Response

1.1 Therapeutic proteins corresponding to Human proteins that include an extra-cellular domain and that have viral homologs, and viral proteins that include an extra-cellular domain and that have human homologs—Such proteins can be secreted or membrane proteins that have an extracellular domain.

Many viruses exploit the strategy of using homologs of cellular cytokines, chemokines, their inhibitors or their receptors to shield virus-infected cells from immune defenses and enhance virus survival in the host. The presence of virus-encoded homologs of cellular proteins may be an indicator of the importance of these cellular components in immune mechanisms for combating this virus in vivo. A number of herpes viruses harbor homologs of IL-10, including Epstein-Barr virus (EBV)-encoded IL-10 (ebvIL-10), (Moore, K. W., Vieira, P., Fiorentino, D. F., Trounstine, M. L., Khan, T. A. & Mosmann, T. R. (1990) Science 248, 1230-1234), and Human cytomegalovirus (CMV)-encoded IL-10 (cmvEL-10) (Proc Natl Acad Sci USA Feb. 15, 2000;97(4):1695-700). These virus-encoded IL-10 homologs downregulate cellular immune responses by suppressing the synthesis of pro-inflammatory cytokines and the ability of macrophages to act as antigen-presenting or co-stimulatory cells. EBV seems to have optimized vIL-10 for immune evasion by retaining the anti-inflammatory, but not the immunostimulatory, characteristics of host IL-10 (reviewed in Nat Rev Immunol. January 2003 ;3(1):36-50).

1.2 Therapeutic proteins corresponding to membrane-anchored human proteins that have secreted viral homologs, in which the extra-cellular domain of the human protein is homologous to the viral protein and membrane-anchored viral proteins that have secreted human homologs, in which the extra-cellular domain of the viral protein is homologous to the human protein—For example, the transcriptionally active open reading frame from Shope Fibroma Virus was shown to have sequence homology with the receptor for human tumor necrosis factor. Since the viral protein possesses a leader sequence but lacks a transmembrane domain, it represents a soluble form of the type I TNF receptor which is secreted from virally infected cells, and whose function is to immunosuppress the host by abrogating the potentially destructive effects of TNF. This was the first such virally-encoded soluble cytokine receptor to be identified, and represents a general mechanism by which viruses subvert the host immune system (Biochem Biophys Res Commun Apr. 15, 1991;176(1):335-42).

1.3 Therapeutic proteins corresponding to human proteins that comprise an intra-cellular domain and that have viral homologs and to viral proteins that comprise an intra-cellular domain and that have Human homologs—Such proteins can be intracellular proteins or membrane proteins that have an intracellular domain. The enhancement in expression or function of this group of therapeutic proteins can be achieved by using gene therapy methods, or by delivering therapeutic proteins to a person in need thereof via vehicles like TAT and liposomes.

The molecular mechanism for the inhibition of the pro-inflammatory response of macrophages during infection by African swine fever virus (ASFV) is an example for this approach of viral modulation of the immune system. The ASFV-encoded protein, A238Lp, which has homology to IκBα, binds directly to the p65 subunit of NFκB. During activation of cells, IκB is targeted for degradation, allowing transport of the NF-κB into the nucleus. The current-accepted model for the action of the viral IκB homologue would be for the viral protein to bind NF-κB but be resistant to signal induced degradation. Essentially, the protein would act as a dominant negative inhibitor of NF-κB by retaining the protein in the cytoplasm (J. Biol. Chem., Vol. 275, Issue 44, 34656-34664, Nov. 3, 2000).

1.4 Therapeutic proteins corresponding to viral proteins that comprise an extra-cellular domain and that share some motifs with a Human protein. Such proteins can be secreted or membrane proteins that have an extracellular domain. An example is the Human Herpesvirus 8 (HHV8)-encoded vMIP-II (virally encoded macrophage inflammatory protein II). This viral protein antagonizes the signaling of six out of the 18 human chemokines receptors (reviewed in Morten Lindow, Hans Rudolf Luttichau and Thue W. Schwartz. Viral leads for chemokine-modulatory drugs. (2003) TRENDS in Pharmacological Sciences 24, 126-130). VMIP-ll share a domain with several cytokines, including CCL3, CCL4, CCL15, CCL23, CCL18, CCL5, CCL14, CCL17, CCL26, CCL22, CCL24, CCL16, CCL13, CCL8, CCL19, CCL1 and others. Another example are the poxviruses encoded growth factors which are related to the mammalian epidermal growth factor (EGF). Growth factors of Shope fibroma virus, Myxoma virus and vaccinia virus (SFGF, MGF and VGF) are capable of binding mammalian ErbB proteins and induce cell proliferation due to attenuation of receptor degradation, which leads to sustained signal transduction (Tzahar E, EMBO J Oct. 15, 1998; 17(20):5948-63).

2. Therapeutic Proteins that Can Act to Block Viral Infections.

2.1 Therapeutic proteins corresponding to human proteins that have a viral homolog, for which the viral homolog lacks a functional domain. These viral-human homologs are believed to have evolved to compete with a corresponding human protein that interferes with some stages in the viral life cycle. The viral homolog is therefore an inactive version of the human protein that is able compete with it for binding to receptors, partners, substrates, etc.

As used herein a “functional domain” refers to a region of a protein sequence, which displays a particular function. This function may give rise to a biological, chemical, or physiological consequence which may be reversible or irreversible and which may include protein-protein interactions (e.g., binding interactions) involving the functional domain, a change in the conformation or a transformation into a different chemical state of the functional domain or of molecules acted upon by the functional domain, the transduction of an intracellular or intercellular signal, the regulation of gene or protein expression, the regulation of cell growth or death, or the activation or inhibition of an immune response.

According to one embodiment, the therapeutic proteins correspond to human proteins that have Pfams domains (http://www.sanger.ac.uk/Software/Pfam/) that do not appear in the homologue viral protein. Identification of functional domains can be effected by comparing a human gene product and it viral homolog with a series of profiles prepared by alignment of well characterized proteins from a number of different species. This generates a consensus profile, which can then be matched with the query sequence. Examples of programs suitable for such identification include, but are not limited to, InterPro Scan—Integrated search in PROSITE, Pfam, PRINTS and other family and domain databases; ScanProsite—Scans a sequence against PROSITE or a pattern against SWISS-PROT and TrEMBL; MotifScan—Scans a sequence against protein profile databases (including PROSITE); Frame-ProfileScan—Scans a short DNA sequence against protein profile databases (including PROSITE); Pfam HMM search—scans a sequence against the Pfam protein families database; FingerPRINTScan—Scans a protein sequence against the PRINTS Protein Fingerprint Database; FPAT—Regular expression searches in protein databases; PRATT—Interactively generates conserved patterns from a series of unaligned proteins; PPSEARCH—Scans a sequence against PROSITE (allows a graphical output); at EBI; PROSITE scan—Scans a sequence against PROSITE (allows mismatches); at PBIL; PATTINPROT—Scans a protein sequence or a protein database for one or several pattern(s); at PBIL; SMART—Simple Modular Architecture Research Tool; at EMBL; TEIRESIAS—Generate patterns from a collection of unaligned protein or DNA sequences; at IBM, all available from http://www.expasy.org/tools/.

3. A method for inhibiting viral infection. Such a method can be effected by silencing or inhibiting the expression or function of a human protein that has a viral homolog, for example through the action of a small molecule and/or by using antisense or siRNA. Thus, these human proteins or their genes serve as drug targets for the development of anti-viral pharmaceuticals. The approach of targeting a host protein to treat viral infections enjoys low rate of viral resistance relative to the approach of targeting viral proteins. Generally, the chances of the virus being able to develop resistance are lower if a human protein is targeted, rather than its viral homology. The rational is that viruses express (mainly) proteins that are needed for their survival. A human homolog for a viral protein implies that this protein is needed for viral survival. Viruses that need excess amounts of such human proteins encode them in their genomes. Other viruses, which do not encode these proteins, might also need these human proteins but in lesser amounts, or may not need them at all. Alternatively, these viruses may use other mechanisms to increase the amount and/or effect of such proteins.

3.1 According to one embodiment, the human proteins have an intra-cellular domain (it may be an intra-cellular protein or a transmembrane protein).

4. A method for inhibiting tumor growth. The method is effected by silencing or inhibiting the expression or function of proteins such as human proteins that have an intra-cellular domain (it may be an intra-cellular protein or a transmembrane protein) and that have a viral homolog. Such proteins are believed to be acting within the cell for function(s) related to viral replication and/or cell proliferation, and therefore to have either a direct or indirect effect on cell growth which may be tumorigenic. Thus, these human proteins serve as drug targets for the development of anti-tumor pharmaceuticals

4.1 According to a preferred embodiment, a relatively high sequence similarity between the human and the viral proteins is required, as this is the case of known oncogenes. For example, c-src is the proto-oncogene corresponding to the oncogene of the src gene of the Rous sarcoma virus (reviewed in Molecular Biology of the Cell, 3rd edn. Part IV. Cells in Their Social Context Chapter 24. Cancer). Silencing or expression inhibition can be effected by gene therapy methods. Alternatively, silencing or expression inhibition is effected by using therapeutic proteins that are delivered to the person in need via vehicles like TAT and liposomes.

It will be appreciated that therapeutic polypeptides and polynucleotides encoding same uncovered using the teachings of the present invention encompass also their biologically functional equivalents, essentially, polynucleotide and polypeptide sequences which include modifications and changes in the structure ( i.e., exhibiting at least 20%, at least 30%, at least 40%, at least 50%, at least 60%, at least 70% , at least 80% , at least 90%, say 95% homology) while still retaining a functional molecule protein with desirable characteristics.

According to one aspect of the present invention there is provided an isolated polynucleotide comprising a nucleic acid sequence encoding a human polypeptide having local homology of at least 20% to a viral polypeptide set forth in the file “patent_virus_info” and “patent_virus_info2” of the enclosed CD-ROM, as determined using the TBlastN software of the National Center of Biotechnology Information (NCBI) using default parameters or as described herein.

According to further features in preferred embodiments of the invention described below, said nucleic acid sequence is set forth in the file “patent_transc_nuc” of the enclosed CD-ROM or in the file “patent_human_transc_{—}2” of enclosed CD-ROM.

According to still further features in the described preferred embodiments the isolated polynucleotide further comprises an additional nucleic acid sequence encoding a label.

According to still further features in the described preferred embodiments said label is selected from the group consisting of an enzymatic label, an oligomerizing label, a fluorescent label and a toxin.

According to another aspect of the present invention there is provided an isolated polynucleotide comprising a nucleic acid sequence of the nucleic acid sequences set forth in the file “patent_transc_nuc” of the enclosed CD-ROM or in the file “patent_human_transc_{—}2” of the enclosed CD-ROM.

According to yet another aspect of the present invention there is provided a pharmaceutical composition comprising a therapeutically effective amount of at least an active portion of a human polypeptide having local homology of at least 20% to a viral polypeptide set forth in the file “patent_virus_info” and “patent_virus_info2” of the enclosed CD-ROM, as determined using the BlastP software of the National Center of Biotechnology Information (NCBI) using default parameters or as described herein and a pharmaceutically acceptable carrier or diluent.

According to still another aspect of the present invention there is provided a pharmaceutical composition comprising a therapeutically effective amount of a polypeptide sequence set forth in the file patent_human_prot_{—}2, or in the file patent_transc_prot, or described in the file patent_virus_info, or described in the file patent_virus_info_{—}2, or described in the file patent_virus_clusters, or described in-the file patent_virus_clusters_{—}2, or of a polynucleotide sequence set forth in the file “patent_transc_nuc”, or in the file patent_human_transc_{—}2 of the enclosed CD-ROM, and a pharmaceutically acceptable carrier or diluent.

According to an additional aspect of the present invention there is provided n isolated polypeptide comprising a human amino acid sequence having local homology of at least 20% to a viral polypeptide set forth in the file “patent_virus_info” and “patent_virus_info2” of the enclosed CD-ROM, as determined using the BlastP software of the National Center of Biotechnology Information (NCBI) using default parameters or as described herein. Preferably, homology of at least about 80% is provided, more preferably at least about 85%, also more preferably at least about 90% and most preferably at least about 95%.

According to still further features in the described preferred embodiments the polypeptide is set forth in the file “patent_transc_prot” or patent_human_prot_{—}2 of the enclosed CD-ROM.

According to yet additional aspect of the present invention there is provided a pharmaceutical composition comprising an amino acid sequence of the viral polypeptides described in the file “”patent_virus_info”, or in the file “patent_virus_info2” or in the file patent_virus_clusters, or in the file patent_virus_clusters_{—}2 of enclosed CD-ROM, and a pharmaceutically acceptable carrier or diluent.

According to still additional aspect of the present invention there is provided a method of modulating an immune response or cell-proliferation in a subject, the method comprising providing to a subject in need thereof a therapeutically effective amount of a human protein having a secreted or an extra-cellular domain, said secreted or extra-cellular domain being at least 20% homologous to a viral protein, as determined using the BlastP software of the National Center of Biotechnology Information (NCBI) using default parameters or as described herein. Preferably, homology of at least about 80% is provided, more preferably at least about 85%, also more preferably at least about 90% and most preferably at least about 95%.

According to still further features in the described preferred embodiments said human protein or viral protein is selected according to at least one sequence criterion set, forth in columns 4, 5, 6 or 7 of file “patent_transc_info” of the enclosed CD-ROM, in file “patent_human_transc_info_{—}2.txt” of enclosed CD-ROM, in columns 5, 6 or 7 of the file “patent_virus_info” of the enclosed CD-ROM and/or in file “patent virus_info_{—}2” of the enclosed CD-ROM, or in the file patent_virus_clusters, or in the file patent_virus_clusters_{—}2 of the enclosed CD-ROM.

According to still further features in the described preferred embodiments said human protein or viral protein is as set forth in any of the sequences in the file “patent_human_transc_{—}2” or in the file “patent_human_prot_{—}2” of the enclosed CD-ROM.

According to still additional aspect of the present invention there is provided a method of modulating an immune response or cell-proliferation in a subject, the method comprising providing to a subject in need thereof a therapeutically effective amount of a secreted viral protein being at least 20% homologous to an extracellular portion of a human protein as determined using the BlastP software of the National Center of Biotechnology Information (NCBI) using default parameters or as described herein. Preferably, homology of at least about 80% is provided, more preferably at least about 85%, also more preferably at least about 90% and most preferably at least about 95%.

According to still further features in the described preferred embodiments said viral protein is described in the file “patent_virus_info” of the enclosed CD-ROM and/or in file “patent_virus_info_{—}2” of the enclosed CD-ROM, or in the file patent_virus_clusters, or in the file patent_virus_clusters_{—}2 of the enclosed CD-ROM.

According to still additional aspect of the present invention there is provided a method of modulating an immune response or cell-proliferation in a subject, the method comprising providing to a subject in need thereof a therapeutically effective amount of:

(i) at least an extracellular domain of a viral protein described in the file “patent_virus_info”, or in the file “patent_virus_info_{—}2” or in the file patent_virus_clusters, or in the file patent_virus_clusters_{—}2 of the enclosed CD-ROM;

(ii) at least an extracellular domain of a human protein, set forth in the file “patent_transc_prot” or patent_human_prot_{—}2.txt of the enclosed CD-ROM; or

(iii) at least an extracellular portion of a membrane-anchored human protein, said extracellular portion being at least 20% homologous to an extracellular portion of a viral protein, as determined using the BlastP software of the National Center of Biotechnology Information (NCBI) using default parameters or as described herein. Preferably, homology of at least about 80% is provided, more preferably at least about 85%, also more preferably at least about 90% and most preferably at least about 95%.

According to still additional aspect of the present invention there is provided a method of modulating an immune response or cell-proliferation in a subject, the method comprises modulating in a subject in need thereof an expression and/or activity of at least one human protein having an intracellular sequence region at least 20% homologous to a viral protein encompassing an intracellular sequence region as determined using the BlastP software of the National Center of Biotechnology Information (NCBI) using default parameters, or as described herein. Preferably, homology of at least about 80% is provided, more preferably at least about 85%, also more preferably at least about 90% and most preferably at least about 95%.

According to still further features in the described preferred embodiments said human protein is selected according to at least one sequence criterion set forth in columns 4, 5, 6 or 7 of file “patent_transc_info” of the enclosed CD-ROM and/or in columns 5, 6 or 7 of file “patent_virus_info” of the enclosed CD-ROM.

According to still further features in the described preferred embodiments said human protein is encoded by any of the nucleic acid sequences set forth in the file “patent_human_transc_{—}2” of the enclosed CD-ROM.

According to still further features in the described preferred embodiments said human protein is set forth in any of the amino acid sequences in the file “patent_human_prot_{—}2” of the enclosed CD-ROM.

According to still further features in the described preferred embodiments said modulating is upregulating.

According to still further features in the described preferred embodiments said upregulating is effected by administering said at least one protein to the subject.

According to still further features in the described preferred embodiments said upregulating is effected by administering an expressible polynucleotide encoding said at least one protein to the subject.

According to still further features in the described preferred embodiments said modulating is downregulating.

According to still further features in the described preferred embodiments said downregulating expression and/or activity of said human protein is effected by an agent selected from the group consisting of:

(i) an oligonucleotide directed to a nucleic acid sequence encoding said human protein;

(ii) a chemical inhibitor directed at said human protein;

(iii) a neutralizing antibody directed at said human protein; and

(iv) a non-functional derivative of said human protein.

According to still additional aspect of the present invention there is provided a method of modulating cell proliferation in a subject, the method comprising downregulating in a subject in need thereof at least one human protein having an intracellular domain which is at least 20% homologous to a viral protein, as determined using the BlastP software of the National Center of Biotechnology Information (NCBI) using default parameters or as described herein.

According to still further features in the described preferred embodiments said at least one human protein is selected according to at least one sequence criterion set forth in columns 4, 5, 6 or 7 of file “patent_transc_info” of the enclosed CD-ROM.

According to still further features in the described preferred embodiments said at least one human membrane-anchored protein is selected according to at least one sequence criterion set forth in columns 4, 5, 6 or 7 of file “patent_transc_info” and/or an E-score lower than 0.00002.

According to still further features in the described preferred embodiments said downregulating is effected by an agent selected from the group consisting of:

(i) an oligonucleotide directed to a nucleic acid sequence encoding said human protein;

(ii) a chemical inhibitor directed to said human protein;

(iii) a neutralizing antibody directed at said human protein; and

(iv) a non-functional derivative of said human protein.

According to still further features in the described preferred embodiments said downregulating is effected by providing to said subject in need thereof a non-functional derivative of said human protein.

According to still further features in the described preferred embodiments said providing is effected by administering said non-functional derivative of said human protein to the subject.

According to still further features in the described preferred embodiments said providing is effected by administering an expressible polynucleotide encoding said non-functional derivative of said human protein.

According to still additional aspect of the present invention there is provided a method of inhibiting a viral infection in a subject, the method comprising providing to a subject in need thereof a therapeutically effective amount of a human protein having an intra-cellular domain having a viral homologue, said viral homologue lacking a functional domain.

According to still further features in the described preferred embodiments said human protein is selected according to at least one sequence criterion set forth in column 6 of file “patent_transc_info” of enclosed CD-ROM.

According to still additional aspect of the present invention there is provided a method of inhibiting a viral infection in a subject, the method comprising providing to a subject in need thereof a therapeutically effective amount of a biomolecule or a small molecule each being capable of binding a human protein having an intra-cellular domain having a viral homolog.

According to still further features in the described preferred embodiments said human protein is selected according to at least one sequence criterion set forth in columns 4, 5, 6 or 7 of file “patent_transc_info” of enclosed CD-ROM or in the file “patent_human_transc_info_{—}2.txt of enclosed CD-ROM.

According to still additional aspect of the present invention there is provided a method of treating immune disorders, tumors and/or metastasis in a subject, the method comprising providing to the subject a 10L biomolecule, fusion homologs or active portions thereof.

According to still additional aspect of the present invention there is provided a method of treating immune disorders, tumors and/or metastasis in a subject, the method comprising providing to the subject a 149R biomolecule, fusions homologs or active portions thereof.

According to still additional aspect of the present invention there is provided a method of treating an immune disorder in a subject, the method comprising providing to the subject a viral complement binding biomolecule, fusions homologs or active portions thereof.

According to still additional aspect of the present invention there is provided a method of treating immune disorders, tumors and/or metastasis in a subject, the method comprising providing to the subject a CD24_HUMAN, fusions homologs, orthologs, or active portions thereof.

According to still further features in the described preferred embodiments said CD24_HUMAN ortholog is a Human herpes virus-5 UL139 biomolecule.

Unless otherwise stated, a nucleotide or amino acid sequence according to the present invention is determined according to homology of at least about 80%, more preferably at least about 85%, also more preferably at least about 90% and most preferably at least about 95%, to at least one sequence described herein and/or given in an attached CD-ROM, as described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Unless otherwise defined, all technical and scientific terms used herein have-the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS:

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows protein sequence comparison between 10L protein [Yaba-like disease virus (gi 12084993) and Serp1 [Myxoma virus] (gi 9633644).

FIG. 2 shows protein sequence comparison between 10 L protein [Yaba-like disease virus] (gi 12084993) and Plasminogen activator inhibitor-1 precursor (PAI-1) (Endothelial plasminogen activator inhibitor) (PAI) (gi 129576).

FIG. 3 shows protein sequence comparison between 149R protein [Yaba-like disease virus] (gi|12085132) and ILEU_HUMAN Leukocyte elastase inhibitor (LEI) (gi|266344)

FIG. 4 shows protein sequence comparison between 149R protein [Yaba-like disease virus] (gi|12085132) and |SCC1_HUMAN Squamous cell carcinoma antigen 1 (gi|20141712)

FIG. 5 shows protein sequence comparison between complement binding protein [Macaca mulatta rhadinovirus] (gi|4494910) and C4BP_HUMAN C4b-binding protein alpha chain precursor (gi|416733)

FIG. 6 shows protein sequence comparison between CD24 GI| 7019343 and UL139 UL139 GI| 29123350

### DESCRIPTION OF THE PREFERRED EMBODIMENTS

The present invention is of a plurality of human-viral homologs having defined properties and/or functions, for particular therapeutic and/or diagnostic applications or uses. The homologs according to the present invention also encompass many novel sequences, as well as previously known sequences for which no diagnostic and/or therapeutic application, and/or a different diagnostic and/or therapeutic application, has been taught or suggested in the background art.

The present inventors have designed a computational approach for high throughput, large scale prediction of human-viral homologs which have defined properties and/or functions and as such may be used in various clinical applications. This computational approach is based on sequence alignment of human transcriptome and proteome to viral databases to thereby find more therapeutic proteins and drug target genes as described above. As described in Examples 1 and 2 of the Examples section, the human genome, the mRNA, and the EST sequence data utilized in this robust sequence analysis were derived from Genbank version 133 (example 1) and version 136 (example 2). Measures were taken not to include viruses which do not infect humans (e.g., insect viruses such as bacullovirus). Typically, homologous sequences may be identified using any sequence alignment software known in the art. Human-virus homologs of the present invention were identified using Tblastn or BlastP alignment software using default parameters or as described in Example 1 and 2 of the Examples section and references therein.

Once human-viral homologs are available, therapeutic proteins (i.e., viral or human) are selected based upon local homology at functional domains thereof (e.g., extracellular domain). Such an analysis is exemplified in Example 2 of the Examples section.

Sequences uncovered as described herein can be experimentally validated using any method known in the art, such as northern blot, RT-PCR, western-blot and the like.

By applying the algorithms described hereinabove and in the Examples section, which follows, the present inventors collected sequence information which is presented in the files “patent_transc_nuc”, “patent_transc_prot.txt”, “patent_human_transc_{—}2” and “patent_human_prot_{—}2” of the enclosed CD-ROM. Novel polynucleotide sequences uncovered using the above-described methodology can be used in various clinical applications (e.g., therapeutic and diagnostic) as is further described hereinbelow.

Sequence information obtained according to the present invention may optionally be used for determining sequences that are suitable for particular therapeutic and/or diagnostic applications or uses. Therapeutic applications or uses may also optionally include being used as a drug and/or antibody target, for example. Therefore, the present invention also encompasses antibodies capable of binding to, and optionally also being elicited by, at least one epitope on a protein or peptide human-virus homolog. Methods of generating antibodies are further described hereinbelow.

Also, the homologs according to the present invention may optionally be used as an entire sequence or as fragments thereof, such as oligonucleotides and/or peptides, and/or nucleic acid fragments and/or partial proteins or protein fragments, for example.

Oligonucleotides, peptides and methods of generating same are described in details hereinbelow.

As described above, some of the therapeutic and/or diagnostic applications of the present invention are related to modulation of the immune system or modulation of cell proliferation with the viral-human homologs. Preferably, such homologs correspond to human proteins that include an extra-cellular domain and that have viral homologs, and viral proteins that include an extra-cellular domain and that have human homologs. Also optionally and preferably, such homologs correspond to membrane-anchored human proteins that have secreted viral homologs, in which the extra-cellular domain of the human protein is homologous to the viral protein and membrane-anchored viral proteins that have secreted human homologs, in which the extra-cellular domain of the viral protein is homologous to the human protein. In a similar but opposing manner, the homologs may optionally correspond to human proteins that comprise an intra-cellular domain and that have viral homologs and to viral proteins that comprise an intra-cellular domain and that have human homologs (see Examples 3-6). The ability to modulate cell proliferation or immune-response protein cascades prompts the use of the proteins (or polynucleotides encoding same) of the present invention for the treatment of cancer and immune disorders, such as described hereinabove.

The present invention also encompasses methods for determining potential drug targets for intervention, for example proteins having an intracellular function that are related to viral proteins which are important for viral replication. Such proteins are downregulated, either at the activity level or at the expression level to inhibit or to treat viral infections or cancer. Methods of inactivating gene expression are further described hereinbelow.

Antibodies

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Inactivation of Gene Expression

RNA interference—RNA interference is a two-step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH MRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with GIC content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

DNAZvme technology—DNAzyme molecules are capable of specifically cleaving an MRNA transcript or DNA sequence of the target sequence. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G.F. Proc. Natl, Acad. Sci. USA 1997;943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G.F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-ab 1 oncogenes were successful in inhibiting the oncogenes expression in leukemia cells, and lessening relapse rates in autologous bone marrow transplant in cases of CML and ALL.

Antisense technology—Downregulation of a target sequence can also be effected by using an antisense oligonucleotide capable of specifically hybridizing with an mRNA transcript of interest.

Design of antisense molecules must be effected while considering two aspects important to the antisense approach. The first aspect is delivery of the oligonucleotide into the cytoplasm of the appropriate cells, while the second aspect is design of an oligonucleotide which specifically binds the designated MRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target MRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp 130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Ribozyme technology—Ribozyme molecules are capable of specifically cleaving an mRNA transcript encoding a specific protein product. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

TFO oligonucleotides—An additional method of regulating the expression of a target sequence in cells is via triplex forming oligonuclotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science,1989;245:725-730; Moser, H. E., et al., Science,1987;238:645-630; Beal, P. A., et al, Science,1992;251:1360-1363; Cooney, M., et al., Science,1988;241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, Sep. 12, 2002, Epub). The same authors have demonstrated that TFOs designed according to the A-AT and G-GC rule do not form non-specific triplexes, indicating that the triplex formation is indeed sequence specific.

Triplex-forming oligonucleotides preferably are at least about 15, more preferably about 25, still more preferably about 30 or more nucleotides in length, up to about 50 or about 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999;27: 1176-81, and Puri, et al, J Biol Chem, 2001;276:28991-98), and the sequence- and target specific downregulation of expression of the Ets2 transcription factor, important in prostate cancer etiology (Carbone, et al, Nucl Acid Res. 2003;31:833-43), and the pro-inflammatory ICAM-1 gene (Besch et al, J Biol Chem, 2002;277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000;28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. patent application Nos. 2003 017068 and 2003 0096980 to Froehler et al, and 2002 0128218 and 2002 0123476 to Emanuele et al, and U.S. Pat. No. 5,721,138 to Lawn.

Oligonucleotides

Oligonucleotides designed for carrying out the methods of the present invention for any of the sequences provided herein (designed as described above) can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, intemucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos: ,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl; or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No: 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No: 3,687,808, those disclosed in The Concise Encyclopedia Of Polymer Science and Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. , ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No: 6,303,374.

It is not necessary for all positions in a given oligonucleotide molecule to be uniformly modified, and in fact more than one of the aforementioned modifications may be incorporated in a single compound or even at a single nucleoside within an oligonucleotide.

Peptides

Peptides of present invention can be biochemically synthesized such as by using standard solid phase techniques. These methods include exclusive solid phase synthesis, partial solid phase synthesis methods, fragment condensation and classical solution synthesis. These methods are preferably used when the peptide is relatively short (i.e., 10 kDa) and/or when it cannot be produced by recombinant techniques (i.e., not encoded by a nucleic acid sequence) and therefore involve different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.], the composition of which can be confirmed via amino acid sequencing and/or mass spectroscopy.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Pharmaceutical Compositions and Administration

Polynucleotides, polypeptides, peptides, antibodies or oligonucleotides of the present invention can be provided to the subject per se, or as part of a pharmaceutical composition where they are mixed with a pharmaceutically acceptable carrier.

A pharmaceutical composition according to the present invention refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally. grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffm, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingi, et al., 1975, in “The Pharmacological Basis of Therapeutics”, Ch. 1 p.1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Description of Sequence Information Uncovered According to the Guidelines of the Present Invention

Viralproteins—All viral proteins (Total 271,202 proteins) were downloaded from NCBI genbank on May 11, 2003. All the HIV-1 proteins were removed and then a non-redundant set was prepared using 95% identity as a cutoff (Holm L, Sander C. Removimg near-neighbour redundancy from large protein sequence collections. Bioinformatics June 1998;14(5):423-9). The new parameters in this run were as follows: fcutoff=0.75; idecutoff=0.95 where the default parameters are: fcutoff=0.5; idecutoff=0.9.

This results in 57,697 proteins. The cluster members of each of the viral proteins are described in table 4.

Human Transcripts—All human transcripts were taken from Gencarta 3.3. This database, which is commercially available from Compugen LTD, is a model of the transcriptome based on the sequences of genbank 133 (Dec. 15, 2002 NCBI-GenBank Flat File Release 133.0).

Human-Virus Homologs—Human-Virus homologs were defined using TBlastn (Altschul S F, Gish W, Miller W, Myers E W, Lipman D J. Basic local alignment search tool. J Mol Biol Oct. 5, 1990;215(3):403-10) using default parameters (Altschul, S. F., Madden, T. L., Schaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402).

Cellular localization—The chances of having a signal peptide in a protein, and the location, if any, of transmembrane segments in proteins was calculated using ProLoc (commercially available from Compugen LTD). Proloc was used for protein subcellular localization prediction.

For this prediction two main approaches were used: (i) the presence of known extracellular domain/s in a protein (as appears in Table 1A; Interpro domains that characterize secreted proteins); (ii) calculating putative transmembrane segments, if any, in the protein and calculating 2 p-values for the existence of a signal peptide. The latest is done by a search for a signal peptide at the N-terminal sequence of the protein generating a score. Running the program on real signal peptides and on N-terminal protein sequences that lack a signal peptide resulted in two score distributions: the first is the score distribution of the real signal peptides and the second is the score distribution of the N-terminal protein sequences that lack the signal peptide. Given a new protein, ProLoc calculates its score and outputs the percentage of the scores that are higher than the current score, in the first distribution, as a first p-value (lower p-values mean more reliable signal peptide prediction) and the percentage of the scores that are lower than the current score, in the second distribution, as a second p-value (lower p-values mean more reliable non signal peptide prediction).

TABLE 1A

| | |
|---|---|
| IPR000874 | Bombesin-like peptide |
| IPR001693 | Calcitonin-like |
| IPR001651 | Gastrin/cholecystokinin peptide hormone |
| IPR000532 | Glucagon/GIP/secretin/VIP |
| IPR001545 | Gonadotropin, beta chain |
| IPR004825 | Insulin/IGF/relaxin |
| IPR000663 | Natriuretic peptide |
| IPR001955 | Pancreatic hormone |
| IPR001400 | Somatotropin hormone |
| IPR002040 | Tachykinin/Neurokinin |
| IPR006081 | Alpha defensin |
| IPR001928 | Endothelin-like toxin |
| IPR001415 | Parathyroid hormone |
| IPR001400 | Somatotropin hormone |
| IPR001990 | Chromogranin/secretogranin |
| IPR001819 | Chromogranin A/B |
| IPR002012 | Gonadotropin-releasing hormone |
| IPR001152 | Thymosin beta-4 |
| IPR000187 | Corticotropin-releasing factor, CRF |
| IPR001545 | Gonadotropin, beta chain |
| IPR000476 | Glycoprotein hormones alpha chain |
| IPR000476 | Glycoprotein hormones alpha chain |
| IPR001323 | Erythropoietin/thrombopoeitin |
| IPR001894 | Cathelicidin |
| IPR001894 | Cathelicidin |
| IPR001483 | Urotensin II |
| IPR006024 | Opioid neuropeptide precursor |
| IPR000020 | Anaphylatoxin/fibulin |
| IPR000074 | Apolipoprotein A1/A4/E |
| IPR001073 | Complement C1q protein |
| IPR000117 | Kappa casein |
| IPR001588 | Casein, alpha/beta |
| IPR001855 | Beta defensin |
| IPR001651 | Gastrin/cholecystokinin peptide hormone |
| IPR000867 | Insulin-like growth factor-binding protein, |
| | IGFBP |
| IPR001811 | Small chemokine, interleukin-8 like |
| IPR004825 | Insulin/IGF/relaxin |
| IPR002350 | Serine protease inhibitor, Kazal type |
| IPR000001 | Kringle |
| IPR002072 | Nerve growth factor |
| IPR001839 | Transforming growth factor beta (TGFb) |
| IPR001111 | Transforming growth factor beta (TGFb), N- |
| | terminal |
| IPR001820 | Tissue inhibitor of metalloproteinase |
| IPR000264 | Serum albumin family |
| IPR005817 | Wnt superfamily |

The identifier is an arbitrary serial number given by the present inventors. Sequence information is described in Tables 1-3, below.

TABLE 1
Human transcript information (32,261 transcripts, not all of them
have a known or predicted protein. file name: ‘patent_transc_info’ of enclosed CD-
ROM)

1^{st} column: Transcript index (example 1.38).
* In this table all the indices start with “1.”.
2^{nd} column: Transcript name (example AA026150_4).
3^{rd} column: RNA name (example rna AK091481).
* If there is no RNA sequence in the transcript than “no rna” appears in this
column.
* If there is a RefSeq sequence in the transcript than “refseq” prefixes the RNA
name.
4^{th} column: SP score (example 0.867784).
* A program that scores an N-terminal peptide of a protein as a signal peptide
was developed. The program was operated on a first group of proteins known to have a
signal peptide, and on a second group of proteins that are known to lack a signal peptide,
resulting in two score distributions. These score distributions were used to calculate two
values (SP score and non SP score) for comparison to the signal peptide score calculated
for each query sequence by the program.
The “SP score” is the percentage of scores that are higher than the score of the
current transcript (query sequence) in the scores distribution of the proteins that have
signal peptide. A lower score is an indication of the reliability of the signal peptide
prediction.
* If no protein is predicted (or known) to be encoded by this transcript then “NO
PROTEIN” appears in this column.
* If the N-terminal peptide is missing in the predicted (or known) protein than
“PROTEIN LACK N_TERMINUS PART” appears in this column.
5^{th} column: Non SP score (example 0.701752).
* The “Non SP score” is the percentage of scores that are lower than the score
of the current transcript (query sequence) in the scores distribution of the proteins that do
not have a signal peptide. A lower score is an indication of the reliability of the prediction
of a lack of a signal peptide.
6^{th} column: Domains (example #IPR IPR000884 #DE Thrombospondin, type I
#IPR IPR001627 #DE Semaphorin/CD100 antigen #IPR IPR001687 #DE ATP/GTP-
binding site motif A (P-loop) #IPR IPR002165 #DE Plexin #IPR IPR003659 #DE
Plexin/semaphorin/integrin).
* #IPR is the name of the domain in the InterPro database.
* #DE is the description of the domain.
* If no domains are found in the protein this column is empty.
7^{th} column: Location of TM segments (example 57-78 1036-1057).
* If putative transmembrane regions are found in the protein their location in the
protein is given.
* If no putative transmembrane regions are found this column is empty.

TABLE 2
Viral protein information (4758 proteins, File name ‘patent_virus_info’
of enclosed CD-ROM)

1^{st} column: Protein index (example 2.5).
* In this table all the indices start with “2.”.
2^{nd} column: Protein accession in genbank (example
gi|10039363|dbj|BAB13332.1|).
3^{rd} column: Name (example Hepatitis B virus).
4^{th} column: Family (example Viruses; Retroid viruses; Hepadnaviridae;
Orthohepadnavirus.).
5^{th} column: SP score (example 0.925026).
6^{th} column: Non SP score (example 0.414098).
7^{th} column: Location of TM segments (example 130-151).

TABLE 3
Human/Virus homologs (260363 hits. E-score varies from 0 to 0.009. File
name ‘patent_pairs_virus_transc’ of enclosed CD-ROM)

1^{st} column: Pair index (example 3.1.2).
* In this table all the indices start with “3.”.
* The second index is for the viral protein and the third index is for each
human transcript that matches the viral protein.
2^{nd} column: Viral protein ID (example gi|1000399|gb|AAB34716.1|).
3^{rd} column: Human transcript ID (example AA179349_1).
4^{th} column: Location of alignment (example VS: 38 VE: 234 HS:
1176 HE: 1715).
* VS: Viral protein start location of the alignment.
* VE: Viral protein end location of the alignment.
* HS: Human transcript start location of the alignment.
* HE: Human transcript end location of the alignment.
5^{th} column: E-score (example 7e−08).

The use of a non-redundant program created clusters of viral proteins that share 95% of similarity. From each cluster one representative viral protein was chosen for this research.

This table shows the other viral proteins in the cluster.

TABLE 4
Viral clusters of proteins (File name ‘patent_virus_clusters’)

* Each line starts with an index (example 4.2.2). All indices in this table
start with “4.”. The second index is the index of the cluster
and the third index is the index of the protein in the cluster.
The first protein in the cluster is the representative.
* Than there is the ID of the viral protein (example
gi|18767712|ref|NP_572003.1|) and a description thereof (example
ribonucleoside-diphosphate reductase beta subunit-like protein
[Rana tigrina ranavirus]).
Sequence information of the above described transcripts is provided in the
following Fasta format files:
(i) File name ‘patent_transc_nuc’ illustrating the sequence
of each of the transcripts described hereinabove;
(ii) File name ‘patent_transc_prot’ illustrating
the predicted or known protein
sequence of each of the transcripts described hereinabove.

### Example 2

### Description of Sequence Information Uncovered According to the Guidelines of the Present Invention

Viralproteins—All viral proteins (Total 294,805 proteins) were downloaded from NCBI genbank on Jan. 10, 2003. All the Baculoviridae and Entomopoxvirinae proteins, which are known to infect only insects, were removed and then a non-redundant set was prepared using 95% identity as a cutoff (Holm L, Sander C. Removing near-neighbour redundancy from large protein sequence collections. Bioinformatics June 1998;14(5):423-9). This results in 97,979 proteins. The cluster members of each of the viral proteins are described in Table 12.

Human Transcripts—All human transcripts were taken from Gencarta 3.4. This database, which is commercially available from Compugen LTD, is a model of the transcriptome based on the sequences of genbank 136.

Human Proteins—All human proteins were taken from Gencarta 3.4. This database, which is commercially available from Compugen LTD, is a model of the transcriptome based on the sequences of genbank 136.

Human-Virus Homologs—Human-Virus homologs were defined using blastp or tblastn (Altschul S F, Gish W, Miller W, Myers E W, Lipman D J. Basic local alignment search tool. J Mol Biol Oct. 5, 1990;215(3):403-10) using default parameters or as described.

Cellular localization—ProLoc, commercially available from Compugen LTD, was used to predict the cellular localization of the human and the viral proteins. Two main approaches have been used: (i) the presence of known extracellular domain/s in a protein; (ii) calculating putative transmembrane segments, if any, in the protein and calculating 2 p-values for the existence of a signal peptide. The latest is done by a search for a signal peptide at the N-terminal sequence of the protein generating a score. Running the program on real signal peptides and on N-terminal protein sequences that lack a signal peptide resulted in 2 scores distributions: the first is the score distribution of the real signal peptides and the second is the score distribution of the N-terminal protein sequences that lack the signal peptide. Given a new protein, ProLoc calculates its score and outputs the percentage of the scores that are higher than the current score, in the first distribution, as a first p-value (lower p-values mean more reliable signal peptide prediction) and the percentage of the scores that are lower than the current score, in the second distribution, as a second p-value (lower p-values mean more reliable non signal peptide prediction).

This invention is related to actively secreted proteins and membrane proteins that have extracellular domain/s. These proteins are identified by ProLoc.

Moreover, this invention is also related to proteins that might be localized outside the cell only in certain conditions (e.g. after lysis of viral-infected cells), and can function outside the cell. These proteins are identified by their sequence homology to proteins that were identified by ProLoc as having extracellular domain/s. The Runs numbered 2 and 4 identify viral proteins that have sequence similarity to human proteins that ProLoc identified as extracellular proteins.

Four different runs—In order to identify secreted viral proteins that may modulate our immune system, the following runs were performed:

Run 1: tblastn of Viral proteins against Human transcripts. Only viral proteins that were predicted to be secreted were used. All human transcripts were used. Maximum E-Score allowed was 0.01.

Debris of viral-infected cells may contain viral proteins that were previously cytoplasmic proteins, and released to the extracellular matrix as a result of the lysis of the infected cell. Moreover, some secreted proteins lack a typical signal peptide and/or domain/s known to be extracellular. To identify such proteins we performed the following:

Run 2: blastp of Human proteins against viral proteins. Only human proteins that were predicted to be secreted were used. All viral proteins were used. Maximum E-Score allowed was 0.01.

Many hormones, cytokines and other important secreted and membrane-bound proteins are known to be post-translationally cleaved. Sometimes, resulting in functional short peptides. Alignment algorithms, like Blast, usually give a relatively low scores for hits of short peptides. In order to facilitate the discovery of hits of short peptide we performed the following two runs:

Run 3: tblastn of Viral proteins against Human transcripts. Only viral proteins that were predicted to be secreted were used. All human transcripts were used. A special amino acid substitution matrix was used, in which the diagonal was 10, and all the rest were -10 (“Identity Matrix“). Gap initiation and extension penalties were both 32,767. Hits were then a subject of Pairwise alignment using the commonly used Blosum62, and gap initiation and extension penalties were set to 9 and 2, respectively. The criteria for a hit were: (i) the Pairwise alignment include the area found by the “Identity Matrix”; (ii) the Pairwise alignment length was shorter than 51 amino acid residues and longer than 7 amino acid residues.

Combining the considerations described for Run No. 2 and Run No. 3, the following was performed:

RUN 4: blastp of Human proteins against viral proteins. Only human proteins that were predicted to be secreted were used. All viral proteins were used. A special amino acid substitution matrix was used, in which the diagonal was 10, and all the rest were −10 (“Identity Matrix”). Gap initiation and extension penalties were both 32,767. Hits were then a subject of Pairwise alignment using the commonly used Blosum62, and gap initiation and extension penalties were set to 9 and 2, respectively. The criteria for a hit were: (i) the Pairwise alignment include the area found by the “Identity Matrix”; (ii) the Pairwise alignment length was shorter than 51 amino acid residues and longer than 7 amino acid residues.

The identifier is an arbitrary serial number given by the present inventors. Sequence information is described in Tables 5-11, below.

TABLE 5
Human transcript information (120,342 transcripts, not all of them
have a known or predicted protein. File name: ‘patent_human_transc_info_2.txt’ of
enclosed CD-ROM)

This is a Tab-delimited file (may consists empty columns).
1^{st} column: Transcript index (example “5.22”).
* In this table all the indices start with “5.”. The following number (in the above
example “22”) is a serial number of human transcript.
2^{nd} column: Transcript internal name (example “AA001480_0”).
3^{rd} column: List of GO annotations, if there are any, of all the proteins in the
transcript's contig (example “#GO_P #GO_Acc 8284 #GO_Desc positive regulation of
cell proliferation #CL 2 #DB sp #EN Q9Y586 #GO_P #GO_Acc 7399 #GO_Desc
neurogenesis #CL 1 #DB sp #EN Q9Y586 #GO_C #GO_Acc 5737 #GO_Desc cytoplasm
#CL 3 #DB PROLOC #EN PROLOC #GO_F #GO_Acc 3824 #GO_Desc catalytic
activity #CL 4 #DB sp #EN Q9Y586”).
* Annotations of transcripts based on protein Gene Ontology (GO) are indicated
by the following format.
** “#GO_P”, annotations related to Biological Process,
** “#GO_F”, annotations related to Molecular Function, and
** “#GO_C”, annotations related to Cellular Component.
For each category the following features are optionally addressed:
** “#GO_Acc” represents the accession number of the assigned GO entry
(“8284” in the above example), corresponding to the following “#GO_Desc”
field.
** “#GO_Desc” represents the description of the assigned GO entry
(“positive regulation of cell proliferation” in the above example), corresponding
to the mentioned “#GO_Acc” field.
** “#CL” represents the confidence level of the GO assignment (“2” in the
above example related to #GO_Acc 8284), when “1” is the highest and “5” is the
lowest possible confidence level.

A preliminary confidence levels were calculated for all public proteins as follows:

PCL 1: a public protein that has a curated GO annotation,

PCL 2: a public protein that has over 85% identity to a public protein with a curated GO annotation,

PCL 3: a public protein that has over 50% identity and less than 85% to a public protein with a curated GO annotation,

PCL 4: a public protein that has under 50% identity to a public protein with a curated GO annotation.

For each Gencarta protein a homology search against all public proteins was done. If the Gencarta protein has over 95% identity to a public protein with PCL X than the Gencarta protein gets the same confidence level as the public protein. This confidence level is marked as “#CL X”. If the Gencarta protein has over 85% identity but not over 95% to a public protein with PCL X than the Gencarta protein gets a confidence level lower by 1 than the confidence level of the public protein. If the Gencarta protein has over 70% identity but not over 85% to a public protein with PCL X than the Gencarta protein gets a confidence level lower by 2 than the confidence level of the public protein. If the Gencarta protein has over 50% identity but not over 70% to a public protein with PCL X than the Gencarta protein gets a confidence level lower by 3 than the confidence level of the public protein. If the Gencarta protein has over 30% identity but not over 50% to a public protein with PCL X than the Gencarta protein gets a confidence level lower by 4 than the confidence level of the public protein.

A Gencarta protein may get confidence level of 2 also if it has a true interpro domain that is linked to a GO annotation http://www.geneontology.org/external2go/interpro2go/.

The GO annotation claimed is the GO annotation as appears in the patent data but when the confidence level is above “1” we claim the GO annotations at higher levels of the GO hierarchy, based upon the value of the confidence level, where such higher levels exist (e.g. for “#CL 3” we claim the GO annotation as appears and the 2 GO annotations above it in the hierarchy).

** “#DB” marks the database on which the GO assignment relies on (“sp” in the above example). The “sp” relates to SwissProt Protein knowledgebase, available from http://www.expasy.ch/sprot/. “InterPro” refers to the InterPro combined database, available from http://www.ebi.ac.uk/interpro/, which contains information regarding protein families, collected from the following databases: SwissProt (http://www.ebi.ac.uk/swissprott), Prosite (http://www.expasy.ch/prosite/), Pfam (http:H/www.sanger.ac.uk/Software/Pfam/), Prints (http://www.bioinfman.ac.uk/dbbrowser/PRINTS/), Prodom (http://prodes.toulouse.inra.fr/prodom/), Smart (http://smart.embl-heidelberg.de/) and Tigrfams (http://www.tigr.org/TIGRFAMs/). “PROLOC” refers to the database resulted from the execution of ProLoc on the proteins.

** “#EN” represents the accession of the entity in the database (#DB), corresponding to the best hit of the predicted protein (In the above example “Q9Y586” means that the GO assignment was based on SwissProt database, while the closest homologue to the assigned protein is depicted in SwissProt entry “Q9Y586”.

* There may be some GO annotations for the same protein or none.

TABLE 6
Human protein information (7,425 transcripts, not all of them have a
known or predicted protein. File name: ‘patent_human_prot_info_2.txt’ of enclosed
CD-ROM)

This is a Tab-delimited file (may consists empty columns).
1^{st} column: Protein index (example “6.64”).
* In this table all the indices start with “6.”. The following number (in the above
example “64”) is a serial number of human protein.
2^{nd} column: Protein internal name (example “AA089855_P7”).
3^{rd} column: Prediction of secretion (e.g. “EXTRACELLULAR”).
* This column appears only when the protein is predicted by ProLoc to be
secreted.
4^{th} column: Signal Peptide existence p-value (according to ProLoc) relative to the
distribution of signal peptides scores of real signal peptides (example “0.350297”).
5^{th} column: Signal Peptide non existence p-value (according to ProLoc) relative to
the distribution of scores of N-terminal protein sequences that lack the signal peptide
(example “0.969873”).
* The above 2 columns appear only in cases that the value of the first p-value is
lower than the value of the second p-value. (This means that the protein looks more like a
secreted protein rather than one that is not secreted).
6^{th} column: Location of TM segments (example “2-22”).
* This column appears only if there is just one predicted transmembrane region,
and it is close to the N-terminus of the protein.
7^{th} column: List of GO annotations of the protein, if there are any (example
“#GO_F #GO_Acc 3795 #GO_Desc antimicrobial peptide activity #CL 5 #DB sp
#EN Q9Y6Z7 #GO_F #GO_Acc 5529 #GO_Desc sugar binding #CL 1 #DB sp #EN
Q9BWP8 #GO_P #GO_Acc 7157 #GO_Desc heterophilic cell adhesion #CL 4 #DB sp
#EN Q9BWP8”
* The GO annotations appear here in the same format as in Table 6 except that
here the “#GO_C” annotations do not appear.

TABLE 7
Viral protein information (22,020 proteins. File name: ‘patent_virus_info_2’
of enclosed CD-ROM)

This is a Tab-delimited file (may consists empty columns).
1^{st} column: Protein index (example “7.386”).
* In this table all the indices start with “7.”. The following number (in the above
example “386”) is a serial number of the viral protein.
2^{nd} column: Protein accession number in genbank (example “1085821”).
3^{rd} column: Protein name (example “Rabies virus”).
4^{th} column: Protein family (example “Viruses; ssRNA negative-strand viruses;
Mononegavirales; Rhabdoviridae; Lyssavirus.”).
5^{rd} column: Sub Cellular prediction (e.g. “CELL_MEMBRANE_ANCHORI”).
* The prediction is by ProLoc.
6^{th} column: Signal Peptide existence p-value (according to ProLoc) relative to the distribution
of signal peptides scores of real signal peptides (example “0.433358”).
7^{th} column: Signal Peptide non existence p-value (according to ProLoc) relative to
the distribution of scores of N-terminal protein sequences that lack the signal peptide
(example “0.956107”).
8^{th} column: Location of TM segments (example “4-25 458-479”).

TABLE 8
Human/Virus homologs (Run No. 1. 54,812 hits. E-score varies from 0 to
0.009. File name: ‘patent_pairs_virus_human_transc_2’ of enclosed CD-ROM)

This is a Tab-delimited file.
1^{st} column: Pair index (example “8.21.99.1”).
* In this table all the indices start with “8.”.
* The second number is the serial number of the viral protein, the third number is the serial
number of the human transcript that matches the viral protein and the fourth
number is the serial number of the blast hit between the 2 sequences.
2^{nd} column: Viral protein name (example “gi|1150663|emb|CAA50956.1|”).
3^{rd} column: Human transcript internal name (example “Z21579_1”).
4^{th} column: Location of alignment (example “VS: 14 VE: 254 HS: 2962 HE: 3696”).
* VS: Viral protein start location of the alignment.
* VE: Viral protein end location of the alignment.
* HS: Human transcript start location of the alignment.
* HE: Human transcript end location of the alignment.
5^{th} column: Blast E-score (example “3e−13”).

TABLE 9
Human/Virus homologs (Run No. 2. 173,944 hits. E-score varies from 0 to
0.009. File name: ‘patent_pairs_virus_human_prot_2’ of enclosed CD-ROM)

This is a Tab-delimited file.
1^{st} column: Pair index (example “9.6.29.1”).
* In this table all the indices start with “9.”.
* The second number is the serial number of the viral protein, the third number is the serial
number of the human protein that matches the viral protein and the fourth
number is the serial number of the blast hit between the 2 sequences.
2^{nd} column: Viral protein name (example “gi|10120606|pdb|1E5G|A”).
3^{rd} column: Human protein internal name (example “HSCR1RS_P8”).
4^{th} column: Location of alignment (example “VS: 3 VE: 120 HS: 104 HE: 234”).
* VS: Viral protein start location of the alignment.
* VE: Viral protein end location of the alignment.
* HS: Human protein start location of the alignment.
* HE: Human protein end location of the alignment.
5^{th} column: Blast E-score (example “1e−17”).

TABLE 10
Human/Virus homologs (Run No. 3. 254,341 hits. Bit-score varies from 9.5
to 78.2. File name: ‘patent_pairs_virus_human_transc_short_proteins_2’ of enclosed
CD-ROM)

This is a Tab-delimited file.
1^{st} column: Pair index (example “10.1.1.1”).
* In this table all the indices start with “10.”.
* The second number is the serial number of the viral protein, the third number is the serial
number of the human transcript that matches the viral protein and the fourth
number is the serial number of the blast hit between the 2 sequences.
2^{nd} column: Viral protein name (example “gi|1000290|gb|AAC54540.1|”).
3^{rd} column: Human transcript internal name (example “AW084614_0”).
4^{th} column: Location of alignment (example “VS:30 VE:37 HS:326 HE:349”).
* VS: Viral protein start location of the alignment.
* VE: Viral protein end location of the alignment.
* HS: Human transcript start location of the alignment.
* HE: Human transcript end location of the alignment.
5^{th} column: Blast Bit-score (example “19.4”).

TABLE 11
Human/Virus homologs (Run No.4. 4,015 hits. Bit-score varies from 10.7 to
42.8. File name: ‘patent_pairs_virus_human_prot_short_proteins_2’ of enclosed CD-
ROM)

This is a Tab-delimited file.
1^{st} column: Pair index (example “11.2.1.1”).
* In this table all the indices start with “11.”.
* The second number is the serial number of the viral protein, the third number is the serial
number of the human protein that matches the viral protein and the fourth
number is the serial number of the blast hit between the 2 sequences.
2^{nd} column: Viral protein name (example “gi|1009267|dbj|BAA07094.1|”).
3^{rd} column: Human protein internal name (example “HSM801067_P1”).
4^{th} column: Location of alignment (example “VS:398 VE:409 HS:58 HE:69”).
* VS: Viral protein start location of the alignment.
* VE: Viral protein end location of the alignment.
* HS: Human protein start location of the alignment.
* HE: Human protein end location of the alignment.
5^{th} column: Blast Bit-score (example “21.8”).

The use of a non redundant program created clusters of viral proteins that share 95% of similarity. From each cluster one representative viral protein was chosen for this research.

This table shows the other viral proteins in the cluster.

TABLE 12
Viral clusters of proteins (File name: ‘patent_virus_clusters_2’)

* Each line starts with an index (example “12.5.3”). All indices in this table start
with “12.”. The second number is the serial number of the cluster and the third number is
the serial number of the protein in the cluster. The first protein in the cluster (i.e. the
proteins numbered 12.x.1, when x is any number) is the representative in the other tables.
* Than the ID of the viral protein (example “gi|871521|emb|CAA68260.1|”) and a
description thereof (example “gag [Avian erythroblastosis virus]”).

Sequence information of the above described human transcripts and proteins are provided in the following Fasta-formatted files.

(i) File name ‘patent_human_transc_{—}2’ illustrating the sequence of each of the transcripts described hereinabove;

(ii) File name ‘patent_human_prot_{—}2’ illustrating the predicted or known human protein sequence of each of the proteins described hereinabove.

### Example 3

The present invention relates to use of a viral protein, 10L, its analogs and biologically active fragments in the prevention and/or treatment of cancer, metastasis and unwanted immune disorders, mainly those in which inflammation play a role. Such conditions include host-versus-graft disease, vascular intimal hyperplasia and restenosis following arterial recanalization intervention procedures, and various autoimmune disorders.

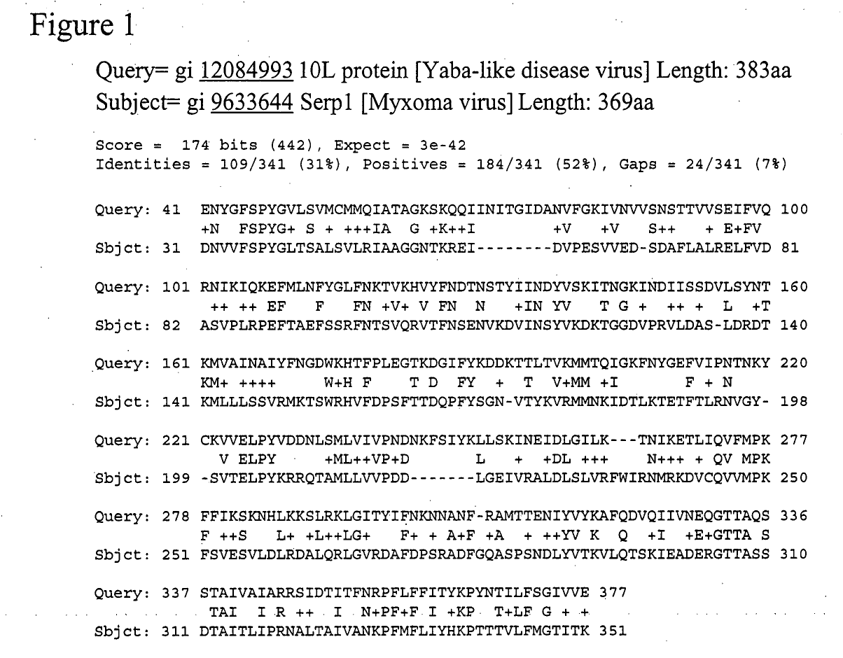

The viral protein 10L is produced according to the genome sequence of Yaba-like disease virus, a member of the Yatapoxvirus. This genome sequence was published (Lee H J, Essani K, Smith G L. The genome sequence of Yaba-like disease virus, a yatapoxvirus. Virology. Mar. 15, 2001;281(2):170-92). The 10L protein shares sequence homology with SERP-1 of Myxoma virus (52% similarity, 31% identity, FIG. 1).

Myxoma virus, a member of Leporipoxvirus, encodes several proteins with anti-immune properties, such as secreted homologues for the cellular receptors for Tumor Necrosis Factor and a serine protease inhibitor, SERP-1 that has demonstrated ability to interfere with the various inflammatory reactions (Bot I, von der Thusen J H, Donners M M, Lucas A, Fekkes M L, de Jager S C, Kuiper J, Daemen M J, van Berkel T J, Heeneman S, Biessen E A. Serine protease inhibitor Serp-1 strongly impairs atherosclerotic lesion formation and induces a stable plaque phenotype in ApoE-/-mice Circ Res. Sep. 5, 2003;93(5):464-71; Dai E, Guan H, Liu L, Little S, McFadden G, Vaziri S, Cao H, Ivanova I A, Bocksch L, Lucas A. Serp-1, a viral anti-inflammatory serpin, regulates cellular serine proteinase and serpin responses to vascular injury. J Biol Chem. May 16, 2003;278(20):18563-72; Hausen B, Boeke K, Berry G J, Morris R E. Viral serine proteinase inhibitor (SERP-1) effectively decreases the incidence of graft vasculopathy in heterotopic heart allografts. Transplantation. Aug. 15, 2001;72(3):364-8; Lucas A, Dai E, Liu L, Guan H, Nash P, McFadden G, Miller L. Transplant vasculopathy: viral anti-inflammatory serpin regulation of atherogenesis. J Heart Lung Transplant. November 2000; 19(11): 1029-38).

More generally, proteins having anti-immune properties that play a role in immunosuppression are produced by some of the large DNA viruses. The state of the art of this field of research is described in two recent reviews (Johnston J B, McFadden G. Poxvirus immunomodulatory strategies: current perspectives. J Virol. June 2003; 77(11):6093-100; Seet B T, Johnston J B, Brunetti C R, Barrett J W, Everett H, Cameron C, Sypula J, Nazarian S H, Lucas A, McFadden G. Poxviruses and immune evasion. Annu Rev Immunol. 2003;21:377423).

SERP-1 is therefore an example of a potential therapeutic protein that can be obtained from such viruses. Furthermore, it was reported that SERP-1 was proven safe when administrated to healthy human in Phase I clinical trials (http:/Hwww.vironinc.com/newsdetails.asp?newsid=14).

According to the present invention, the protein 10L is believed to have immunomodulatory activity and to be able to serve as a therapeutic protein for treating various inflammatory and other immune disorders. Furthermore, 10L may also optionally have a potential use as a therapeutic protein for the treatment of cancer and metastasis. This belief is based at least partially upon the sequence homology of the 10L protein with SERP-1. In addition, the 10L protein has high homology with human protein sequences.

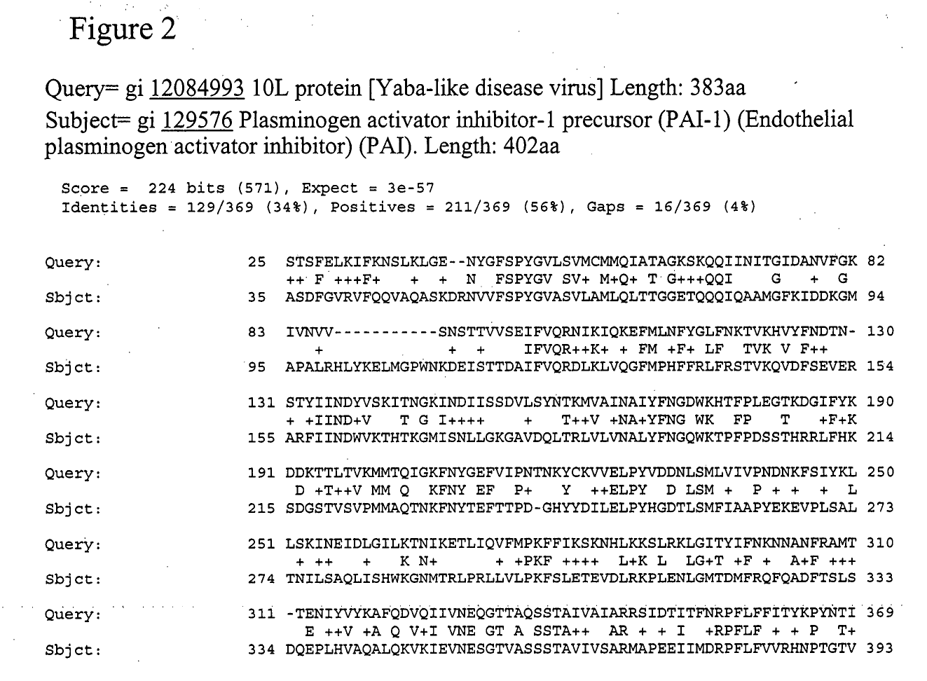

For example, 10L also shares relatively high sequence similarity with human Plasminogen activator inhibitor-1 (PAI-1) (56% similarity, 34% identity, FIG. 2). PAI-1 is a serine proteinase inhibitor in the serpin superfamily (Ny T, Sawdey M, Lawrence D, Millan J L, Loskutoff D J. Cloning and sequence of a cDNA coding for the human beta-migrating endothelial-cell-type plasminogen activator inhibitor. Proc Natl Acad Sci USA 1986, 83:6776-80; Pannekoek H, Veerman H, Lambers H, Diergaarde P, Verweij Q L, van Zonneveld A J, van Mourik J A: Endothelial plasminogen activator inhibitor (PAI): a new member of the Serpin gene family. EMBO J 1986, 5:2539-44; Bermd R. Binder Günter Christ, Florian Gruber, Nelly Grubic, Peter Hufnagl, Michael Krebs, Judit Mihaly and Gerald W. Prager: Plasminogen Activator Inhibitor 1: Physiological and Pathophysiological Roles. News Physiol Sci 2002, 17:56-61). This 50 kDa glycoprotein is apparently the most important physiological inhibitor of tissue-type plasminogen activator and of urokinase plasminogen activator (Loskutoff D J, Schleef R R: Plasminogen activators and their inhibitors. Methods Enzymol 1988, 163:293-302.).

It was shown to play a crucial role in the regulation of vascular thrombosis, tumor invasion, neovascularization, inflammation and wound healing (Andreasen P A Egelund R, Petersen H H: The plasminogen activation system in tumor growth, invasion, and metastasis. Cell Mol Life Sci 2000, 57:25-40; Kohler H P Grant P J: Plasminogen-activator inhibitor type I and coronary artery disease. N Engl J Med 2000, 342:1792-1801).

Furthermore, the specificity of SERPINs is mainly determined by their Reactive Center Loop (RCL, also known as Reactive Site Loop (RSL).). The length of the RCL of Serpins is usually 17 residues. Inhibitory serpins have a consensus pattern in their RCL:

| P17 | P16 | P15 | P14 | P12-P9 |
|---|---|---|---|---|
| E | E/K/R | G | T/S | (A/G/S)₄ |

Inhibitory specificity is considered to depend mainly on residues that flank the site in the RCL that is cleaved upon reaction with the proteinase, mainly with regard to position 1, but also with regard to position 2, and to a lesser extent with regard to position 3. This specificity of Serpins was investigated in many articles for example see: (Cooper S T, Church F C. Reactive site mutants of recombinant protein C inhibitor. Biochim Biophys Acta. Jan. 5, 1995;1246(1):29-33; Djie M Z, Le Bonniec B F, Hopkins P C, Hipler K, Stone S R. Role of the P2 residue in determining the specificity of serpins. Biochemistry. Sep. 3, 1996;35(35):11461-9; Chen V C, Chao L, Chao J. Roles of the P1, P2, and P3 residues in determining inhibitory specificity of kallistatin toward human tissue kallikrein. J Biol Chem. Dec. 8, 2000;275(49):38457-66).

| | |
|---|---|
| 17 12 9 321 1′ | |
| EQGTTAQSSTAIVAIAR RSIDTITF | YLDV 10L |
| ESGTVASSSTAVIVSAR MAPEEIIM | Human PAI-1 |
| ERGTTASSDTAITLIPR NALTAIVA | Myxoma virus SERP-1 |

Thus, sequence comparison of the RCL suggests that the inhibitory specificity of 10L [YLDV] is similar to that of human PAI-1 and SERP-1 [Mv]. 10L protein [Yaba-like disease virus] gi|12056169|emb|CAC21248.1|[12056169] is described in Tables 2 and 7, and its cluster member (gi|12084993) appears in tables 4 and 12.

In Table 9, the 10 best human protein hits for 10L are:

| | | | | |
|---|---|---|---|---|
| 9.202.73.1 | gi\|12084993\|ref\|NP_073395.1\| | T10920_P17 | VS:25 VE:378 HS:35 HE:402 | 7e−53 |
| 9.202.72.1 | gi\|12084993\|ref\|NP_073395.1\| | T10920_P16 | VS:25 VE:378 HS:35 HE:402 | 1e−48 |
| 9.202.54.1 | gi\|12084993\|ref\|NP_073395.1\| | HUMGDN_P6 | VS:1 VE:378 HS:7 HE:398 | 6e−46 |
| 9.202.51.1 | gi\|12084993\|ref\|NP_073395.1\| | HUMGDN_P1 | VS:1 VE:378 HS:7 HE:397 | 1e−45 |
| 9.202.52.1 | gi\|12084993\|ref\|NP_073395.1\| | HUMGDN_P2 | VS:1 VE:378 HS:19 HE:409 | 1e−45 |
| 9.202.53.1 | gi\|12084993\|ref\|NP_073395.1\| | HUMGDN_P3 | VS:1 VE:378 HS:61 HE:451 | 1e−45 |
| 9.202.7.1 | gi\|12084993\|ref\|NP_073395.1\| | F07041_P1 | VS:1 VE:383 HS:1 HE:402 | 8e−39 |
| 9.202.5.1 | gi\|12084993\|ref\|NP_073395.1\| | AF130470_P2 | VS:23 VE:382 HS:24 HE:396 | 4e−35 |
| 9.202.6.1 | gi\|12084993\|ref\|NP_073395.1\| | AF130470_P3 | VS:23 VE:382 HS:24 HE:381 | 1e−34 |
| 9.202.56.1 | gi\|12084993\|ref\|NP_073395.1\| | HUMMNEI_P1 | VS:21 VE:378 HS:4 HE:379 | 8e−28 |

### Example 4

The present invention relates to use of a viral protein, 149R (GI|12056308), its analogs and biologically active fragments in the prevention and/or treatment of cancer, metastasis and unwanted immune disorders, mainly those in which inflammation play a role. Such conditions include host-versus-graft disease, vascular intimal hyperplasia and restenosis following arterial recanalization intervention procedures, and various autoirnmune disorders.

The viral protein 149R is also encoded by the Yaba-like disease virus genome, which was described above with regard to Example 3.

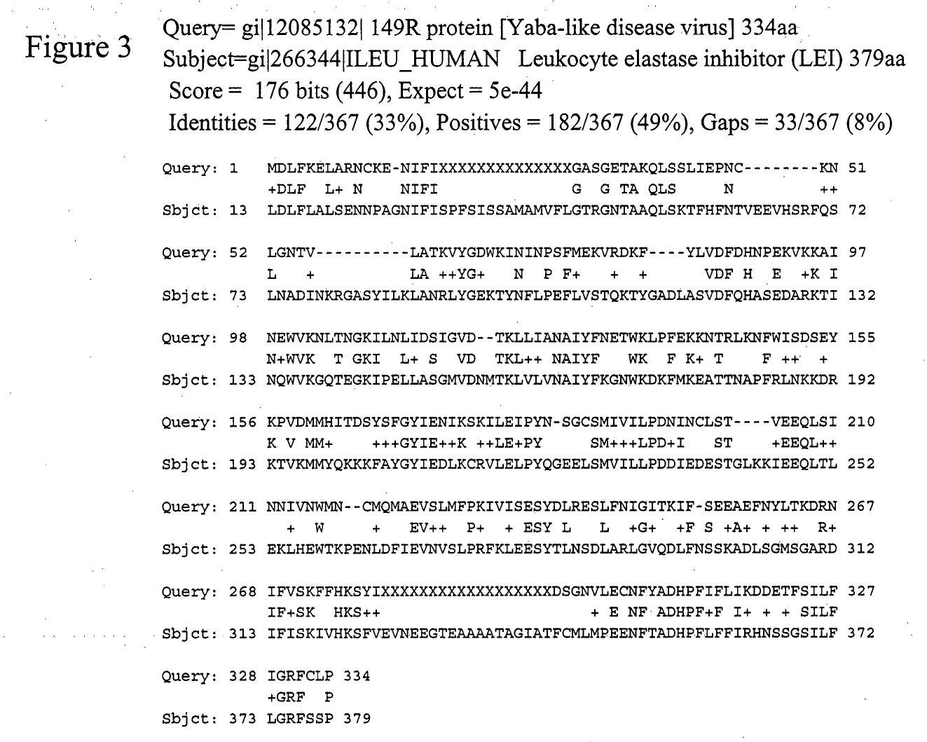

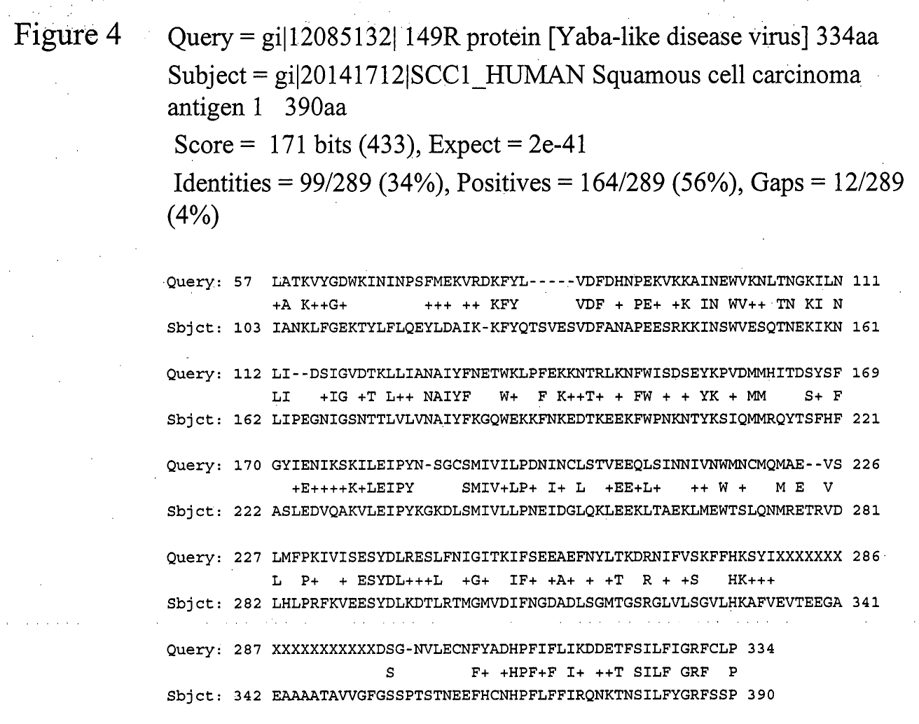

One of its proteins, named 149R, shares sequence homology with human leukocyte elastase (49% similarity, 33% identity, FIG.3) and with human SCCA1 ((56% similarity and 34% identity, FIG.4). These human proteins are serine proteinase inhibitors that belong to the serpin superfamily.

Most programs for prediction of signal peptides in proteins fail to detect signal peptides in 149R, human leukocyte elastase inhibitor, and human SCCA1. However, experimental evidence supports the secretion of SCCA1 (Int. J. Cancer (Pred. Oncol.): 89, 368-377 (2000); and reviewed in Oncol Rep. March-April 2001;8(2):347-54). Furthermore, several reports suggest the ability of recombinant human leukocyte elastase inhibitor to treat inflammatory-based disorders (Am. J. Respir. Cell Mol. Biol., Volume 20, Number 1, January, 1999 69-78. Recombinant Human Monocyte/Neutrophil Elastase Inhibitor Protects Rat Lungs against Injury from Cystic Fibrosis Airway Secretions. Dianne D. Rees, Rick A. Rogers, Jessica Cooley, Robert J. Mandle, Dianne M. Kenney, and Eileen Remold-O'Donnell). Based on its high homology to these human proteins, 149R is believed to be useful as a therapeutic protein, regardless of whether it is naturally secreted.

Leukocyte elastase is a protease that is involved in the tissue destruction and inflammation that characterize numerous diseases, including hereditary emphysema, chronic obstructive pulmonary disease, cystic fibrosis, adult respiratory distress syndrome, ischemic-reperfusion injury and rheumatoid arthritis. Thus, elastase has been the object of extensive research to develop potent inhibitors that target its destructive and pro-inflammatory action. Currently, inhibitors of neutrophil elastase are being developed for their anti-inflammatory activity, including purified or recombinantly produced endogenous inhibitors, genetically modified recombinant protein inhibitors and synthetic small-molecule inhibitors (reviewed in Tremblay G M, Janelle M F, Bourbonnais Y. Anti-inflammatory activity of neutrophil elastase inhibitors. Curr Opin Investig Drugs. May 2003; 4(5):556-65).

According to the present invention, 149R is suggested as a useful therapeutic protein with regard to its potential immunomodulatory activity and its potential ability to serve as a therapeutic protein for treating various inflammatory and other immune disorders.

149R protein [Yaba-like disease virus] gi|12056308|emb|CAC21387.1|[12056308] is described in Tables 2 and 7, and its cluster member (gi|12085132) appears in tables 4 and 12.

In Table 9, the top human protein 10 hits are:

| | | | | |
|---|---|---|---|---|
| 9.186.60.1 | gi\|12056308\|emb\|CAC21387.1\| | HUMMNEI_P1 | VS:1 VE:334 HS:13 HE:379 | 3e−53 |
| 9.186.61.1 | gi\|12056308\|emb\|CAC21387.1\| | HUMMNEI_P6 | VS:1 VE:334 HS:13 HE:372 | 9e−52 |
| 9.186.41.1 | gi\|12056308\|emb\|CAC21387.1\| | HSTHRINH_P5 | VS:1 VE:334 HS:31 HE:394 | 2e−46 |
| 9.186.6.1 | gi\|12056308\|emb\|CAC21387.1\| | AF130470_P3 | VS:1 VE:334 HS:31 HE:377 | 5e−42 |
| 9.186.5.1 | gi\|12056308\|emb\|CAC21387.1\| | AF130470_P2 | VS:1 VE:334 HS:31 HE:392 | 4e−41 |
| 9.186.7.1 | gi\|12056308\|emb\|CAC21387.1\| | F07041_P1 | VS:13 VE:334 HS:44 HE:397 | 2e−40 |
| 9.186.55.1 | gi\|12056308\|emb\|CAC21387.1\| | HUMGDN_P1 | VS:13 VE:334 HS:49 HE:397 | 8e−37 |
| 9.186.56.1 | gi\|12056308\|emb\|CAC21387.1\| | HUMGDN_P2 | VS:13 VE:334 HS:61 HE:409 | 8e−37 |
| 9.186.57.1 | gi\|12056308\|emb\|CAC21387.1\| | HUMGDN_P3 | VS:13 VE:334 HS:103 HE:451 | 9e−37 |
| 9.186.77.1 | gi\|12056308\|emb\|CAC21387.1\| | R83168_P7 | VS:3 VE:334 HS:50 HE:404 | 7e−36 |

In Table 8, the top 10 hits are:

| | | | | |
|---|---|---|---|---|
| 8.40.13.1 | gi\|12056308\|emb\|CAC21387.1\| | AW802938_1 | VS:2 VE:334 HS:158 HE:1042 | 2e−48 |
| 8.40.117.1 | gi\|12056308\|emb\|CAC21387.1\| | HUMMNEI_0 | VS:1 VE:334 HS:240 HE:1139 | 5e−46 |
| 8.40.118.1 | gi\|12056308\|emb\|CAC21387.1\| | HUMMNEI_1 | VS:1 VE:334 HS:240 HE:1139 | 5e−46 |
| 8.40.129.1 | gi\|12056308\|emb\|CAC21387.1\| | HUMMNEI_2 | VS:1 VE:334 HS:240 HE:1139 | 5e−46 |
| 8.40.132.1 | gi\|12056308\|emb\|CAC21387.1\| | HUMMNEI_3 | VS:1 VE:334 HS:240 HE:1139 | 5e−46 |
| 8.40.133.1 | gi\|12056308\|emb\|CAC21387.1\| | HUMMNEI_4 | VS:1 VE:334 HS:240 HE:1139 | 5e−46 |
| 8.40.134.1 | gi\|12056308\|emb\|CAC21387.1\| | HUMMNEI_5 | VS:1 VE:334 HS:500 HE:1039 | 5e−46 |
| 8.40.135.1 | gi\|12056308\|emb\|CAC21387.1\| | HUMMNEI_6 | VS:1 VE:334 HS:1457 HE:2557 | 5e−46 |
| 8.40.136.1 | gi\|12056308\|emb\|CAC21387.1\| | HUMMNEI_7 | VS:1 VE:334 HS:1121 HE:2221 | 5e−46 |
| 8.40.137.1 | gi\|12056308\|emb\|CAC21387.1\| | HUMMNEI_8 | VS:1 VE:334 HS:455 HE:1174 | 5e−46 |

In Table 10, the top 10 hits are:

| | | | | |
|---|---|---|---|---|
| 10.982.10.1 | gi\|12056308\|emb\|CAC21387.1\| | HSCOLLIG_22 | VS:307 VE:334 HS:323 HE:406 | 45.6 |
| 10.982.12.1 | gi\|12056308\|emb\|CAC21387.1\| | T05055_8 | VS:299 VE:317 HS:3331 HE:3384 | 25.1 |
| 10.982.8.1 | gi\|12056308\|emb\|CAC21387.1\| | CD172251_0 | VS:171 VE:204 HS:20 HE:121 | 22.2 |
| 10.982.7.1 | gi\|12056308\|emb\|CAC21387.1\| | C17944_0 | VS:5 VE:14 HS:45 HE:16 | 22.2 |
| 10.982.3.1 | gi\|12056308\|emb\|CAC21387.1\| | AK056835_0 | VS:165 VE:172 HS:615 HE:638 | 21.8 |
| 10.982.1.1 | gi\|12056308\|emb\|CAC21387.1\| | AI355819_0 | VS:165 VE:172 HS:39 HE:62 | 21.8 |
| 10.982.5.1 | gi\|12056308\|emb\|CAC21387.1\| | AW468416_0 | VS:173 VE:182 HS:121 HE:92 | 21.0 |
| 10.982.13.1 | gi\|12056308\|emb\|CAC21387.1\| | Z25122_3 | VS:51 VE:60 HS:923 HE:894 | 20.6 |
| 10.982.15.1 | gi\|12056308\|emb\|CAC21387.1\| | Z41099_1 | VS:171 VE:181 HS:5635 HE:5667 | 20.2 |
| 10.982.14.1 | gi\|12056308\|emb\|CAC21387.1\| | Z38803_0 | VS:51 VE:58 HS:526 HE:549 | 19.8 |

### Example 5

### Complement Binding Protein [Macaca mulatta Rhadinovirus].

The complement system plays a fundamental role in both the innate and acquired immune responses. As such, it also participates in the majority of diseases characterized by acute and/or chronic inflammation. For example, a critical role of the complement system has been demonstrated in rheumatoid arthritis, post-myocardial infarction reperfusion injury, post-bowel ischernia reperfusion injury, and systemic lupus erythematosus. These specific disorders are simply representative of most inflammatory states in which similar or identical molecular pathways result in complement activation and concomitant tissue injury.

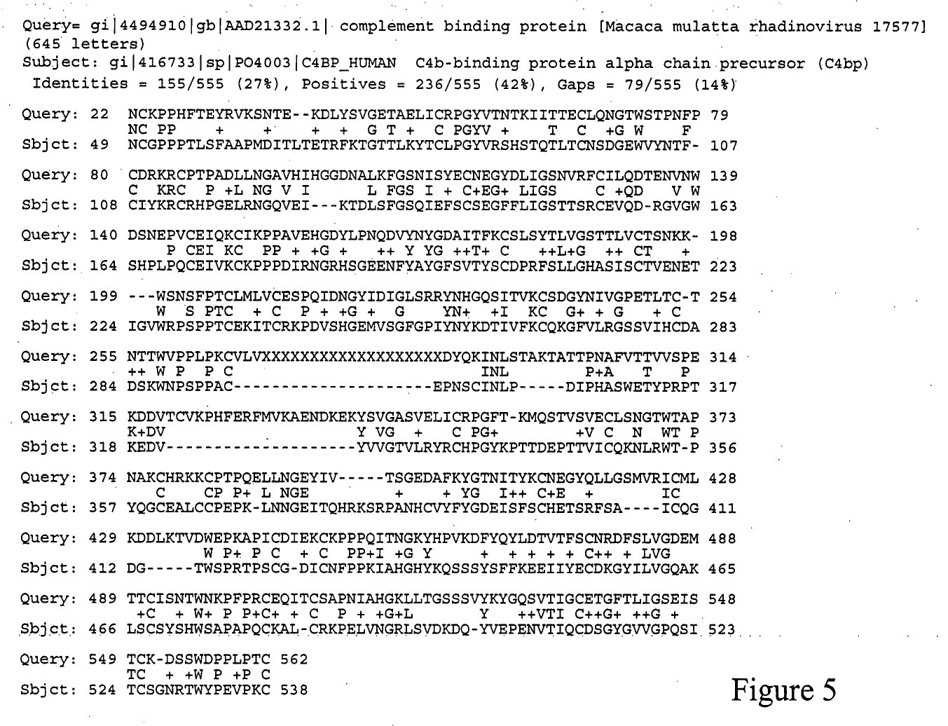

Hyperacute rejection of xenografts has also been shown to result from activation of the human complement system. The utilization of organs obtained from nonhuman donors is an appealing solution to the increasing shortage of organs available for clinical transplantation. Although xenotransplantation using organs obtained from primate donors has been performed with limited clinical success, the use of distantly related species, such as pigs or sheep, avoids ethical dilemmas, potential virus transmission, and limited availability associated with the use of primates as xenograft donors. However, the use of organs from distantly related species for xenotransplantation is impractical due to hyperacute rejection (hyperacute rejection), a process that leads to irreversible xenograft damage and organ loss within minutes to hours. In xenotransplantation of vascularized tissues, hyperacute rejection is thought to be mediated by the binding of naturally occurring recipient antibodies to the endothelium. of the xenograft. The present invention relates to use of a viral protein, complement binding protein of Macaca mulatta rhadinovirus (GI:4494910), its analogs and biologically active fragments in the prevention and/or treatment of unwanted immune disorders, mainly those in which inflammation play a role. This viral protein has a high sequence similarity with human C4b-BP (27% identity; 42% similarity; FIG. 5), which is known to block the complement cascades (reviewed in Complement therapeutics; history and current progress. Morgan B P, Harris C L. Mol Immunol. September 2003;40(2-4):159-70). gi|4494910| complement binding protein [Macaca mulatta rhadinovirus 17577] is described in Tables 2 and 7, and its cluster member (gi|18653812) appears in tables 4 and 12.

In Table 9, the top 10 human protein hits of complement binding protein [Macaca mulatta rhadinovirus 17577] are:

| | | | | |
|---|---|---|---|---|
| 9.2970.30.1 | gi\|4494910\|gb\|AAD21332.1\| | HSCRIRS_P4 | VS:18 VE:562 HS:497 HE:1004 | 3e−54 |
| 9.2970.31.1 | gi\|4494910\|gb\|AAD21332.1\| | HSCRIRS_P5 | VS:23 VE:562 HS:48 HE:554 | 3e−54 |
| 9.2970.34.1 | gi\|4494910\|gb\|AAD21332.1\| | HSCRIRS_P8 | VS:18 VE:562 HS:492 HE:999 | 3e−54 |
| 9.2970.33.1 | gi\|4494910\|gb\|AAD21332.1\| | HSCRIRS_P7 | VS:18 VE:562 HS:492 HE:999 | 9e−54 |
| 9.2970.30.2 | gi\|4494910\|gb\|AAD21332.1\| | HSCRIRS_P4 | VS:18 VE:562 HS:947 HE:1454 | 1e−53 |
| 9.2970.30.3 | gi\|4494910\|gb\|AAD21332.1\| | HSCRIRS_P4 | VS:23 VE:562 HS:48 HE:554 | 1e−53 |
| 9.2970.33.2 | gi\|4494910\|gb\|AAD21332.1\| | HSCRIRS_P7 | VS:23 VE:562 HS:43 HE:549 | 1e−53 |
| 9.2970.34.2 | gi\|4494910\|gb\|AAD21332.1\| | HSCRIRS_P8 | VS:18 VE:562 HS:942 HE:1449 | 1e−53 |
| 9.2970.34.3 | gi\|4494910\|gb\|AAD21332.1\| | HSCRIRS_P8 | VS:23 VE:562 HS:43 HE:549 | 1e−53 |
| 9.2970.85.1 | gi\|4494910\|gb\|AAD21332.1\| | HUMEB2CR2_P8 | VS:52 VE:569 HS:183 HE:664 | 7e−53 |

### Example 6

### UL139 Protein [Human Herpesvirus 5]

The present invention relates to the use of a viral protein, UL139 (GI|29123350), its analogs and biologically active fragments in the prevention and/or treatment of cancer, metastasis and unwanted immune disorders such as Multiple Sclerosis. This alignment is a result of run 5 (see above)

Run 5

Smith-Waterman of Human proteins against viral proteins using default parameters, except that the PAM50 substitution matrix and gap extension penalty of 10 were used. Only human proteins that were predicted to be secreted were used. All viral proteins were used.

Background and Results

Human Cytomegalovirus (HHV-5) encodes a protein with an unknown function, named UL139 (Cha T A, Tom E, Kemble G W, Duke G M, Mocarski E S, Spaete R R, Human cytomegalovirus clinical isolates carry at least 19 genes not found in laboratory strains. J Virol. January 1996;70(1):78-83). Programs for prediction of signal peptides in proteins (e.g. SignalP Server, Henrik Nielsen, Jacob Engelbrecht, Soren Brunak and Gunnar von Heijne: Identification of prokaryotic and eukaryotic signal peptides and prediction of their cleavage sites. Protein Engineering, 10, 1-6 (1997)) detect signal peptide in UL139.

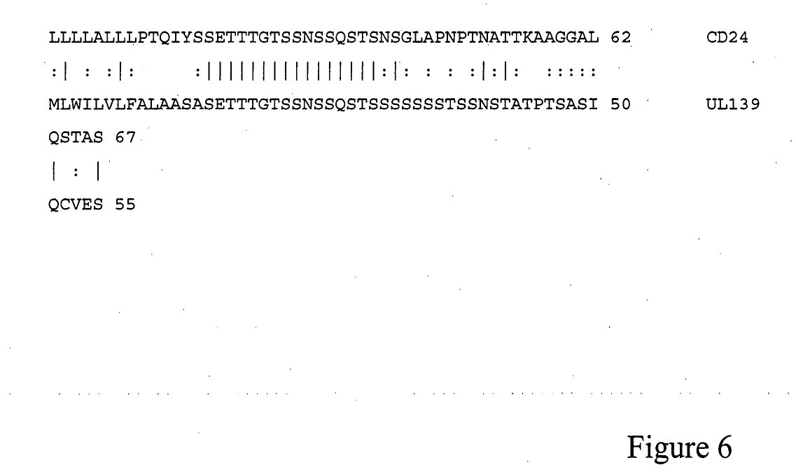

Herein, we show that UL139 of HHV-5 shares sequence homology with HUMAN CD24 (GI|7019343). The similarity appears in a restricted region (see FIG. 6).

CD24 is a cell surface glycoprotein observed in a variety of human malignancies (Kristiansen G, Schluns K, Yongwei Y, Denkert C, Dietel M, Petersen I, CD24 is an independent prognostic marker of survival in nonsmall cell lung cancer patients, Br J Cancer. Jan. 27, 2003;88(2):231-6.). It has no transmembrane segment and is known to attach the cell membrane via a glycosyl phosphatidylinositol (GPI) anchor (Kay R, Rosten P M, Humphries R K. CD24, a signal transducer modulating B cell activation responses, is a very short peptide with a glycosyl phosphatidylinositol membrane anchor, J Immnunol. Aug. 15, 1991;147(4):1412-6).

According to the present invention, UL139 has immunomodulatory activity and can optionally serve as a therapeutic protein for treating various inflammatory and other immune disorders. Furthermore, UL139 may optionally be used for the treatment of cancer and metastasis.

The fragment of UL139 that shares homology with CD24 may also optionally be used for treating various inflammatory and other immune disorders, and as a therapeutic peptide for the treatment of cancer and metastasis.

The present invention also includes the use of the fragment of CD24 that shares homology with UL139 for treating various inflammatory and other immune disorders, and as a therapeutic peptide for the treatment of cancer and metastasis.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### CD-ROM Content

The following duplicate CD-ROM is attached herewith:

File information is provided as: File name/ bite size/date of creation/machine format/operating system.

1. patent_pairs_virus_transc/37,300 Kbytes/Jun. 19, 2003/Internet explorer/PC.

2. patent_transc_nuc/128,534 bytes/Jun. 5, 2003/Internet explorer/PC.

3. patent_virus_clusters/1,431 Kbytes/Jun. 9, 2003/Internet explorer/PC.

4. patent_transc_info/4,681 Kbytes/Jun. 11, 2003/Internet explorer/PC.

5. patent_transc_prot.txt/18,420 Kbytes/Jun. 2, 2003/Internet explorer/PC.

6. patent_virus_info.txt/737 Kbytes/Jun. 11, 2003/Internet explorer/PC.

7. patent_pairs_virus_human_transc_{—}2/4,481 Kbytes/Nov. 20, 2003/Internet explorer/PC.

8. patent_pairs_virus_human_prot_{—}2/14,380 Kbytes/Nov. 20, 2003/Internet explorer/PC.

9. patent_pairs_virus_human_transc_shortproteins_{—}2/20,624 Kbytes/Nov. 20, 2003/Internet explorer/PC.

10. patent_pairs_virus_human_prot short_proteins_{—}2/325 Kbytes/Nov. 20, 2003/Internet explorer/PC.

11. patent_human_transc_info_{—}2.txt/66,968 Kbytes/Dec. 16, 2003/Internet explorer/PC.

12. patent_human_prot_info_{—}2.txt/2,135 Kbytes/Dec. 16, 2003/Internet explorer/PC.

13. patent_virus_info_{—}2/3,459 Kbytes/Nov. 20, 2003/Internet explorer/PC.

14. patent_virus_clusters_{—}2/5,441 Kbytes/Nov. 20, 2003/Internet explorer/PC.

15. patent_human_transc_{—}2/225,402 Kbytes/Nov. 9, 2003/Internet explorer/PC.

16. patent_human_prot_{—}2/4,067 Kbytes/Nov. 9, 2003/Internet explorer/PC.

17. Viral genes.txt/1 Kbytes/Jan. 6, 2004/Internet explorer/PC.

What is claimed is:

1. An isolated polynucleotide comprising a nucleic acid sequence encoding a human polypeptide having local homology of at least 20% to a viral polypeptide set forth in the file “patent_virus_info” and “patent_virus_info2“of the enclosed CD-ROM, as determined using the BlastP software of the National Center of Biotechnology Information (NCBI) using default parameters or as described herein.

2. The isolated polynucleotide of claim 1, wherein said nucleic acid sequence is set forth in the file “patent_transc_nuc” of the enclosed CD-ROM or in the file “patent_human_transc_{—}2” of enclosed CD-ROM.

3. The isolated polynucleotide of claim 1, further comprising an additional nucleic acid sequence encoding a label.

4. The isolated polynucleotide of claim 3, wherein said label is selected from the group consisting of an enzymatic label, an oligomerizing label, a fluorescent label and a toxin.

5. An isolated polynucleotide comprising a nucleic acid sequence of the nucleic acid sequences set forth in the file “patent_transc_nuc” of the enclosed CD-ROM or in the file “patent_human_transc_{—}2” of the enclosed CD-ROM.

6. The isolated polynucleotide of claim 5, further comprising an additional nucleic acid sequence encoding a label.

7. The isolated polynucleotide of claim 6, wherein said label is selected from the group consisting of an enzymatic label, an oligomerizing label, a fluorescent label and a toxin.

8. A pharmaceutical composition comprising a therapeutically effective amount of at least an active portion of a human polypeptide having local homology of at least 20% to a viral polypeptide set forth in the file “patent_virus_info” and “patent_virus_info2“of the enclosed CD-ROM, as determined using the BlastP software of the National Center of Biotechnology Information (NCBI) using default parameters or as described herein and a pharmaceutically acceptable carrier or diluent.

9. The pharmaceutical composition of claim 8, wherein said human polypeptide is set forth in the file patent_human_prot_{—}2 or patent_transc_prot of the enclosed CD-ROM.

10. A pharmaceutical composition comprising a therapeutically effective amount of a polypeptide sequence set forth in the file patent_human_prot_{—}2, or in the file patent_transc_prot, or described in the file patent_virus_info, or described in the file patent_virus_info_{—}2, or described in the file patent_virus_clusters, or described in the file patent_virus_clusters_{—}2, or of a polynucleotide sequence set forth in the file “patent_transc_nuc”, or in the file patent_human_transc_{—}2 of the enclosed CD-ROM, and a pharmaceutically acceptable carrier or diluent.

11. An isolated polypeptide comprising a human amino acid sequence having local homology of at least 20% to a viral polypeptide set forth in the file “patent_virus_info” and “patent_virus_info2” of the enclosed CD-ROM, as determined using the BlastP software of the National Center of Biotechnology Information (NCBI) using default parameters or as described herein.

12. The isolated polypeptide of claim 11, wherein the polypeptide is set forth in the file “patent_transc_prot” or patent_human_prot_{—}2 of the enclosed CD-ROM.

13. A pharmaceutical composition comprising an amino acid sequence of the viral polypeptides described in the file “”patent_virus_info”, or in the file “patent_virus_info2” or in the file patent_virus_clusters, or in the file patent_virus_clusters_{—}2 of enclosed CD-ROM, and a pharmaceutically acceptable carrier or diluent.

14. A method of modulating an immune response or cell-proliferation in a subject, the method comprising providing to a subject in need thereof a therapeutically effective amount of a human protein having a secreted or an extra-cellular domain, said secreted or extra-cellular domain being at least 20% homologous to a viral protein, as determined using the BlastP software of the National Center of Biotechnology Information (NCBI) using default parameters or as described herein.

15. The method of claim 14, wherein said human protein or viral protein is selected according to at least one sequence criterion set forth in columns 4, 5, 6 or 7 of file “patent_transc_info” of the enclosed CD-ROM, in file “patent_human_transc_info_{—}2.txt” of enclosed CD-ROM, in columns 5, 6 or 7 of the file “patent_virus_info” of the enclosed CD-ROM and/or in file “patent_virus_info_{—}2” of the enclosed CD-ROM, or in the file patent_virus_clusters, or in the file patent_virus_clusters_{—}2 of the enclosed CD-ROM.

16. The method of claim 14, wherein said human protein or viral protein is as set forth in any of the sequences in the file “patent_human_transc_{—}2” or in the file “patent_human_prot_{—}2” of the enclosed CD-ROM.

17. A method of modulating an immune response or cell-proliferation in a subject, the method comprising providing to a subject in need thereof a therapeutically effective amount of a secreted viral protein being at least 20% homologous to an extracellular portion of a human protein as determined using the BlastP software of the National Center of Biotechnology Information (NCBI) using default parameters or as described herein.

18. The method of claim 17, wherein said viral protein is described in the file “patent_virus_info” of the enclosed CD-ROM and/or in file “patent_virus_info 2” of the enclosed CD-ROM, or in the file patent_virus_clusters, or in the file patent_virus_clusters_{—}2 of the enclosed CD-ROM.

19. A method of modulating an immune response or cell-proliferation in a subject, the method comprising providing to a subject in need thereof a therapeutically effective amount of:
(i) at least an extracellular domain of a viral protein described in the file “patent_virus_info_{—}2” or in the file patent virus_clusters, or in the file patent_virus_clusters_{—}2 of the enclosed CD-ROM;
(ii) at least an extracellular domain of a human protein, set forth in the file “patent_transc_prot” or patent_human_prot_{—}2.txt of the enclosed CD-ROM; or
(iii) at least an extracellular portion of a membrane-anchored human protein, said extracellular portion being at least 20% homologous to an extracellular portion of a viral protein, as determined using the Blast software of the National Center of Biotechnology Information (NCBI) using default parameters or as described herein

20. The method of claim 19, wherein said human protein is encoded by any of the nucleic acid sequences set forth in the file “patent_human_transc_{—}2” of the enclosed CD-ROM.

21. The method of claim 19, wherein said human protein is set forth in any of the amino acid sequences set forth in the file “patent human_prot_{—}2” of the enclosed CD-ROM.

22. The method of claim 19, wherein said viral protein is described in the file “patent_virus_info_{—}2” of enclosed CD-ROM.

23. A method of modulating an immune response or cell-proliferation in a subject, the method comprises modulating in a subject in need thereof an expression and/or activity of at least one human protein having an intracellular sequence region at least 20% homologous to a viral protein encompassing an intracellular sequence region as determined using the BlastP software of the National Center of Biotechnology Information (NCBI) using default parameters, or as described herein.

24. The method of claim 23, wherein said human protein is selected according to at least one sequence criterion set forth in columns 4, 5, 6 or 7 of file “patent_transc_info” of the enclosed CD-ROM and/or in columns 5, 6 or 7 of file “patent_virus_info” of the enclosed CD-ROM.

25. The method of claim 23, wherein said human protein is encoded by any of the nucleic acid sequences set forth in the file “patent_human_transc_{—}2” of the enclosed CD-ROM.

26. The method of claim 23, wherein said human protein is set forth in any of the amino acid sequences in the file “patent_human_prot_{—}2” of the enclosed CD-ROM.

27. The method of claim 23, wherein said modulating is upregulating.

28. The method of claim 27, wherein said upregulating is effected by administering said at least one protein to the subject.

29. The method of claim 27, wherein said upregulating is effected by administering an expressible polynucleotide encoding said at least one protein to the subject.

30. The method of claim 23, wherein said modulating is downregulating.

31. The method of claim 30, wherein said downregulating expression and/or activity of said human protein is effected by an agent selected from the group consisting of:
(i) an oligonucleotide directed to a nucleic acid sequence encoding said human protein;
(ii) a chemical-inhibitor directed at said human protein;
(iii) a neutralizing antibody directed at said human protein; and
(iv) a non-functional derivative of said human protein.

32. A method of modulating cell proliferation in a subject, the method comprising downregulating in a subject in need thereof at least one human protein having an intracellular domain which is at least 20% homologous to a viral proteini as determined using the BlastP software of the National Center of Biotechnology Information (NCBI) using default parameters or as described herein.

33. The method of claim 32, wherein said at least one human protein is selected according to at least one sequence criterion set forth in columns 4, 5, 6 or 7 of file “patent_transc_info” of the enclosed CD-ROM.

34. The method according to claim 32, said at least one human membrane-anchored protein is selected according to at least one sequence criterion set forth in columns 4, 5, 6 or 7 of file “patent_transc_info” and/or an E-score lower than 0.00002.

35. The method of claim 32, wherein said downregulating is effected by an agent seleceted from the group consisting of:
(i) an oligonucleotide directed to a nucleic acid sequence encoding said human protein;
(ii) a chemical inhibitor directed to said human protein;
(iii) a neutralizing antibody directed at said human protein; and
(iv) a non-functional derivative of said human protein.

36. The method of claim 35, wherein said downregulating is effected by providing to said subject in need thereof a non-functional derivative of said human protein.

37. The method of claim 36, wherein said providing is effected by administering said non-functional derivative of said human protein to the subject.

38. The method of claim 36, wherein said providing is effected by administering an expressible polynucleotide encoding said non-functional derivative of said human protein.

39. A method of inhibiting a viral infection in a subject, the method comprising providing to a subject in need thereof a therapeutically effective amount of a human protein having an intra-cellular domain having a viral homologue, said viral homologue lacking a functional domain.

40. The method of claim 39, wherein said human protein is selected according to at least one sequence criterion set forth in column 6 of file “patent_transc_info” of enclosed CD-ROM.

41. A method of inhibiting a viral infection in a subject, the method comprising providing to a subject in need thereof a therapeutically effective amount of a biomolecule or a small molecule each being capable of binding a human protein having an intra-cellular domain having a viral homolog.

42. The method of claim 41, wherein said human protein is selected according to at least one sequence criterion set forth in columns 4, 5, 6 or 7 of file “patent_transc_info” of enclosed CD-ROM or in the file “patent_human_transc_info_{—}2.txt of enclosed CD-ROM.

43. A method of treating immune disorders, tumors and/or metastasis in a subject, the method comprising providing to the subject a 10L biomolecule, fusion homologs or active portions thereof.

44. A method of treating immune disorders, tumors and/or metastasis in a subject, the method comprising providing to the subject a 149R biomolecule, fusions homologs or active portions thereof.

45. A method of treating an immune disorder in a subject, the method comprising providing to the subject a viral complement binding biomolecule, fusions homologs or active portions thereof.

46. A method of treating immune disorders, tumors and/or metastasis in a subject, the method comprising providing to the subject a CD24_HUMAN, fusions homologs, orthologs, or active portions thereof.

47. The method of claim 46, wherein said CD24_HUMAN ortholog is a Human herpes virus-5 UL139 biomolecule.

48. A method of treating an immune disorder or cancer in a subject the method comprising providing to a subject in need thereof a therapeutically effective amount of a human protein having a secreted or an extra-cellular domain, said secreted or extra-cellular domain being at least 20% homologous to a viral protein, as determined using the BlastP software of the National Center of Biotechnology Information (NCBI) using default parameters or as described herein, thereby treating the immune disorder or cancer in the subject.

49. The method of claim 48, wherein said human protein or viral protein is selected according to at least one sequence criterion set forth in columns 4, 5, 6 or 7 of file “patent_transc_info” of the enclosed CD-ROM, in file “patent_human_transc_info_{—}2.txt” of enclosed CD-ROM, in columns 5, 6 or 7 of the file “patent_virus_info” of the enclosed CD-ROM and/or in file “patent_virus_info_{—}2” of the enclosed CD-ROM, or in the file patent_virus_clusters, or in the file patent_virus_clusters_{—}2 of the enclosed CD-ROM.

50. The method of claim 48, wherein said human protein or viral protein is as set forth in any of the sequences in the file “patent_human_transc_{—}2” or in the file “patent_human_prot_{—}2” of the enclosed CD-ROM.

51. A method of treating an immune disorder or cancer in a subject the method comprising, providing to a subject in need thereof a therapeutically effective amount of a secreted viral protein being at least 20% homologous to an extracellular portion of a human protein as determined using the BlastP software of the National Center of Biotechnology Information (NCBI) using default parameters or as described herein, thereby treating the immune disorder or cancer in the subject.

52. The method of claim 51, wherein said viral protein is described in the file “patent_virus_info” of the enclosed CD-ROM and/or in file “patent_virus_info_{—}2” of the enclosed CD-ROM, or in the file patent_virus_clusters, or in the file patent_virus_clusters 2 of the enclosed CD-ROM.

53. A method of treating an immune disorder or cancer in a subject, the method comprising providing to a subject in need thereof a therapeutically effective amount of:
(i) at least an extracellular domain of a viral protein described in the file “patent_virus_info_{—}2” or in the file patent_virus_clusters, or in the file patent_virus_clusters_{—}2 of the enclosed CD-ROM;
(ii) at least an extracellular domain of a human protein, set forth in the file “patent_transc_prot” or patent_human_prot_{—}2.txt of the enclosed CD-ROM; or
(iii) at least an extracellular portion of a membrane-anchored human protein, said extracellular portion being at least 20% homologous to an extracellular portion of a viral protein, as determined using the Blast software of the National Center of Biotechnology Information (NCBI) using default parameters or as described herein, thereby treating the immune disorder or cancer in the subject.

54. The method of claim 53, wherein said human protein is encoded by any of the nucleic acid sequences set forth in the file “patent_human_transc_{—}2” of the enclosed CD-ROM.

55. The method of claim 53, wherein said human protein is set forth in any of the amino acid sequences set forth in the file “patent_human_prot_{—}2” of the enclosed CD-ROM.

56. The method of claim 53, wherein said viral protein is described in the file “patent_{— virus}_info_{—}2” of enclosed CD-ROM.

57. A method of treating an immune disorder or cancer in a subject, the method comprising modulating in a subject in need thereof an expression and/or activity of at least one human protein having an intracellular sequence region at least 20% homologous to a viral protein encompassing an intracellular sequence region, as determined using the BlastP software of the National Center of Biotechnology Information (NCBI) using default parameters, or as described herein, thereby treating the immune disorder or cancer in a subject.

58. The method of claim 57, wherein said human protein is selected according to at least one sequence criterion set forth in columns 4, 5, 6 or 7 of file “patent_transc_info” of the enclosed. CD-ROM and/or in columns 5, 6 or 7 of file “patent_virus_info” of the enclosed CD-ROM.

59. The method of claim 57, wherein said human protein is encoded by any of the nucleic acid sequences set forth in the file “patent_human_transc_{—}2” of the enclosed CD-ROM.

60. The method of claim 57, wherein said human protein is set forth in any of the amino acid sequences in the file “patent_human_prot_{—}2” of the enclosed CD-ROM.

61. The method of claim 57, wherein said modulating is upregulating.

62. The method of claim 61, wherein said upregulating is effected by administering said at least one protein to the subject.

63. The method of claim 61, wherein said upregulating is effected by administering an expressible polynucleotide encoding said at least one protein to the subject.

64. The method of claim 57, wherein said modulating is downregulating.

65. The method of claim 64, wherein said downregulating expression and/or activity of said human protein is effected by an agent selected from the group consisting of:
(i) an oligonucleotide directed to a nucleic acid sequence encoding said human protein;
(ii) a chemical inhibitor directed at said human protein;
(iii) a neutralizing antibody directed at said human protein; and
(iv) a non-functional derivative of said human protein.

66. A method of treating cancer in a subject, the method comprising downregulating in a subject in need thereof at least one human protein having an intracellular domain which is at least 20% homologous to a viral protein, as determined using the BlastP software of the National Center of Biotechnology Information (NCBI) using default parameters or as described herein, thereby treating the cancer in the subject.

67. The method of claim 66, wherein said at least one human protein is selected according to at least one sequence criterion set forth in columns 4, 5, 6 or 7 of file “patent_transc_info” of the enclosed CD-ROM.

68. The method according to claim 66, said at least one human membrane-anchored protein is selected according to at least one sequence criterion set forth in columns 4, 5, 6 or 7 of file “patent_transc_info” and/or an E-score lower than 0.00002.

69. The method of claim 66, wherein said downregulating is effected by an agent seleceted from the group consisting of:
(i) an oligonucleotide directed to a nucleic acid sequence encoding said human protein;
(ii) a chemical inhibitor directed to said human protein;
(iii) a neutralizing antibody directed at said human protein; and
(iv) a non-functional derivative of said human protein.

70. The method of claim 69, wherein said downregulating is effected by providing to said subject in need thereof a non-functional derivative of said human protein.

71. The method of claim 70, wherein said providing is effected by administering said non-functional derivative of said human protein to the subject.

72. The method of claim 70, wherein said providing is effected by administering an expressible polynucleotide encoding said non-functional derivative of said human protein.

73. A method of treating or preventing viral infection in a subject, the method comprising providing to a subject in need thereof a therapeutically effective amount of a human protein having an intra-cellular domain having a viral homologue, said viral homologue lacking a functional domain, thereby treating or preventing the viral infection in the subject.

74. The method of claim 73, wherein said human protein is selected according to at least one sequence criterion set forth in column 6 of file “patent_transc_info” of enclosed CD-ROM.

75. A method of treating or preventing viral infection in a subject, the method comprising providing to a subject in need thereof a therapeutically effective amount of a biomolecule or a small molecule each being capable of binding a human protein having an intra-cellular domain having a viral homolog, thereby of treating or preventing viral infection in the subject.

76. The method of claim 75, wherein said human protein is selected according to at least one sequence criterion set forth in columns 4, 5, 6 or 7 of file “patent_transc_info” of enclosed CD-ROM or in the file “patent_human_transc_info_{—}2.txt of enclosed CD-ROM.
